(19) United States
(12) Patent Application Publication
(10) Pub. No.: US 2022/0022947 A1
(43) Pub. Date: Jan. 27, 2022

(54) ELECTROCHEMICAL THERAPY OF CANCEROUS TUMORS BASED ON INTRA-THERAPEUTICAL IMPEDANCE MONITORING

(71) Applicants: Mohammad Abdolahad, Tehran (IR); Reyhaneh Mahdavi, Tehran (IR); Mohammad Ali Khayamian, Tehran (IR); Parisa Aghaee, Tehran (IR); Hadi Ghafari, Tehran (IR); Naser Namdar Habashi, Tehran (IR); Fereshteh Abbasvandi, Tehran (IR); Zohreh Sadat Miripour, Tehran (IR); Narges Yousefpour, Tehran (IR); Hossein Ataee, Tehran (IR); Sajad Mehrvarz, Tehran (IR)

(72) Inventors: Mohammad Abdolahad, Tehran (IR); Reyhaneh Mahdavi, Tehran (IR); Mohammad Ali Khayamian, Tehran (IR); Parisa Aghaee, Tehran (IR); Hadi Ghafari, Tehran (IR); Naser Namdar Habashi, Tehran (IR); Fereshteh Abbasvandi, Tehran (IR); Zohreh Sadat Miripour, Tehran (IR); Narges Yousefpour, Tehran (IR); Hossein Ataee, Tehran (IR); Sajad Mehrvarz, Tehran (IR)

(73) Assignee: Nano Hesgarsazan Salamat Arya, Tehran (IR)

(21) Appl. No.: 17/492,659

(22) Filed: Oct. 3, 2021

Related U.S. Application Data
(60) Provisional application No. 63/105,213, filed on Oct. 24, 2020.
(60) Provisional application No. 63/087,183, filed on Oct. 3, 2020.

(51) Int. Cl. A61B 18/14

(52) U.S. Cl. CPC A61B 18/1477; A61B 2018/1266

(57) ABSTRACT

A method for destroying a cancerous tumor. The method includes putting two electrodes of an electrical probe in contact with a portion of the cancerous tumor, plotting an impedance phase diagram by measuring a set of electrical impedance phase values from the portion of the cancerous tumor at end of a respective set of pre-determined time steps, destroying cancer cells of the portion of the cancerous tumor within each time step of the respective set of pre-determined time steps by electrolyzing peripheral medium surrounding the cancer cells of the portion of the cancerous tumor by applying a direct current (DC) voltage between the two electrodes, and stopping destroying of the cancer cells responsive to a complete destruction of the portion of the cancerous tumor, where the complete destruction includes obtaining a positive slope of the impedance phase diagram (IPS).

### CROSS-REFERENCE TO RELATED APPLICATION

This application claims the benefit of priority from pending U.S. Provisional Patent Application Ser. No. 63/087,183 filed on Oct. 3, 2020, and entitled “BIOELECTRICAL PATHOLOGY OF THE BREAST” and pending U.S. Provisional Patent Application Ser. No. 63/105,213 filed on Oct. 24, 2020, and entitled “BIOPSY-FREE CANCER DIAGNOSTIC NEEDLE FOR REAL-TIME DISTINGUISHMENT OF BENIGN AND MALIGNANT BREAST MASSES WITH BI-RADS AND PATHOLOGICAL CALIBRATIONS”, which are both incorporated herein by reference in their entirety.

### TECHNICAL FIELD

The present disclosure generally relates to cancer treatment, and particularly, to electrochemical therapy (EChT) utilizing a needle probe with integrated electrodes.

### BACKGROUND

Electrochemical therapy (EChT) is a well-known tumor destructing method based on electrochemical pH changing of a tumor with electrical direct currents. Up to now, numerous research about EChT have been published, such as a large number of clinical trials as far it shed new light in superficial tumor treatments after its initiation in China in 1987. However, common EChT is unable to treat deep tumoral masses and causes damages due to utilizing Direct Current (DC) fields on skin. Moreover, treating a deep tumoral mass by EChT requires placing cathode and anode electrodes out of a tumor in order to better production of electrolyzing field for complete tumor destruction, which causes side effects on healthy tissues around the tumor. Moreover, there is an absence of a standard methodology for EChT treatments adaptable to different types, locations, and sizes of tumors. Additionally, sonography-assisted monitoring of progression of tumor removal during EChT is impossible due to production of gas bubbles during electrolysis process. The produced gas bubbles make it difficult to utilize any imaging-based monitoring technique. Due to these mentioned disadvantages and limitations, only a few limited medical communities in countries such as China and Germany utilize EChT in rare types of superficial tumors that are non-dissectible by surgery.

Hence, there is a need for devices, systems, and methods for conducting EChT intra-therapeutically for tumor removal of deeply located cancerous tumors. Furthermore, there is a need for devices, systems, and methods for EChT of tumors precisely inside tumor mass and without any side effects on surrounding normal tissues. Moreover, there is a need for devices, systems, and methods allowing for precise monitoring of tumor removal progression while applying EChT.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a method for destroying a cancerous tumor. The method may include putting two electrodes of an electrical probe in contact with a portion of the cancerous tumor, plotting an impedance phase diagram by measuring a set of electrical impedance phase values from the portion of the cancerous tumor at a respective set of pre-determined time steps by applying an alternating current (AC) voltage in a sweeping range of frequencies to the two electrodes at end of each time step of the respective set of pre-determined time steps utilizing an impedance analyzer device, destroying cancer cells of the portion of the cancerous tumor within each time step of the respective set of pre-determined time steps by electrolyzing peripheral medium surrounding the cancer cells of the portion of the cancerous tumor, and stopping destroying of the cancer cells responsive to a complete destruction of the portion of the cancerous tumor, the complete destruction comprising obtaining a positive slope of the impedance phase diagram (IPS) by analyzing the IPS at end of each time step. In an exemplary implementation, the electrolyzing peripheral medium may include applying a direct current (DC) voltage between the two electrodes utilizing a DC voltage generator. In an exemplary implementation, destroying cancer cells of the portion of the cancerous tumor within each time step, plotting the impedance phase diagram at end of each time step, and analyzing the IPS at end of each time step may be done in a cycle iteratively after each other.

In an exemplary implementation, analyzing the IPS at end of each time step may include calculating the IPS at end of each time step, comparing the calculated IPS with zero, and detecting the complete destruction of the portion of the cancerous tumor if the calculated IPS is a positive IPS (IPS value is more than zero). In an exemplary implementation, calculating the IPS at end of each time step may be done using a definition that may include:

$$IPS = \frac{{Phase}_{2} - {Phase}_{1}}{log ( {Frequency}_{2} ) - log ( {Frequency}_{1} )} ,$$

Where, Phase₂ may be a measured impedance phase value at frequency value of Frequency₂ and Phase₁ may be a measured impedance phase value at frequency value of Frequency₁. In an exemplary implementation, stopping destroying of the cancer cells may include ceasing applying the DC voltage between the two electrodes if a value of the calculated IPS is more than zero.

In an exemplary implementation, measuring the set of electrical impedance phase values from the portion of the cancerous tumor at end of each time step may include connecting the two electrodes of the electrical probe to the impedance analyzer device, applying an AC voltage in a sweeping range of frequencies between 1 Hz and 1 MHz to the two electrodes, measuring the set of electrical impedance phase values respective to the swept range of frequencies, and plotting the measured set of electrical impedance phase values versus the swept range of frequencies.

In an exemplary implementation, electrolyzing peripheral medium surrounding the cancer cells of the portion of the cancerous tumor may include connecting the two electrodes of the electrical probe to the DC voltage generator and applying a DC voltage with a magnitude between 0.5 V and 10 V between the two electrodes during each time step utilizing the DC voltage generator.

In an exemplary implementation, destroying cancer cells of the portion of the cancerous tumor within each time step may further include vacuum suction of dead cells. In an exemplary implementation, the vacuum suction of dead cells may include connecting a vacuum pump to a proximal end of the electrical probe and driving out the dead cells from the portion of the cancerous tumor by applying a vacuum pressure more than 20 KPa to the proximal end of the electrical probe.

In an exemplary implementation, the set of pre-determined time steps may include at least one of a set of equal time steps, a set of unequal time steps, and combinations thereof. In an exemplary implementation, each time step of the set of pre-determined time steps may include a time period in a range between 15 seconds and 3 minutes.

In an exemplary implementation, putting the two electrodes of the electrical probe in contact with the portion of the cancerous tumor may include inserting a distal end portion of a first electrode of the electrical probe into the portion of the cancerous tumor and pushing a distal end portion of a second electrode of the electrical probe through the first needle electrode into the portion of the cancerous tumor. In an exemplary embodiment, the first electrode may include a first electrically conductive needle comprising a hollow needle and the second electrode may include a second electrically conductive needle with a nanoporous surface placed inside the first electrode.

In an exemplary implementation, putting the two electrodes of the electrical probe in contact with the portion of the cancerous tumor may further include identifying a location of the portion of the cancerous tumor by at least one of monitoring inserting the distal end portion of the first electrode of the electrical probe into the portion of the cancerous tumor with guide of sonography imaging, comparing an electrical impedance value of a location of the inserted distal end portion of the first electrode with a reference impedance value, and combinations thereof.

In an exemplary implementation, identifying the location of the portion of the cancerous tumor by comparing the electrical impedance value of the portion of location of the inserted distal end portion of the first electrode with the reference impedance value may include connecting the two electrodes of the electrical probe to the impedance analyzer device, applying an AC voltage in a sweeping range of frequencies between 1 Hz and 1 MHz to the two electrodes, measuring an electrical impedance value of the location of the inserted distal end portion of the first electrode at a reference frequency value, and identifying location of the inserted distal end portion of the first electrode as the location of the portion of the cancerous tumor if the measured electrical impedance value is less than the reference impedance value. In an exemplary embodiment, the reference frequency value may include a frequency value of 1 kHz. In an exemplary embodiment, the reference impedance value may include an impedance value of 1500Ω.

In an exemplary implementation, the method may further include preparing the electrical probe. In an exemplary implementation, preparing the electrical probe may include generating a nanoporous surface on an outer surface of the second electrically conductive needle, coating a second electrically insulating layer on the second electrically conductive needle except a distal end portion of the second electrically conductive needle, placing the second electrically conductive needle inside the first electrically conductive needle, covering an outer surface of the first electrically conductive needle with a first electrically insulating layer except a distal end portion of the first electrically conductive needle, and attaching two electrical connectors to the first electrically conductive needle and the second electrically conductive needle. In an exemplary implementation, attaching the two electrical connectors to the first electrically conductive needle and the second electrically conductive needle may include attaching a first electrical connector onto a surface of the first electrically conductive needle adjacent to a proximal end of the first electrically conductive needle and attaching a second electrical connector onto a proximal end of the second electrically conductive needle.

In an exemplary implementation, generating the nanoporous surface on the outer surface of the second electrically conductive needle may include forming a plurality of nanopores. In an exemplary embodiment, each of the nanopores may be a nanopore with a diameter of less than 300 nm.

In an exemplary implementation, generating the nanoporous surface on the outer surface of the second electrically conductive needle may include wet etching of the outer surface of the second electrically conductive needle. In an exemplary implementation, wet etching of the outer surface of the second electrically conductive needle may include placing the second electrically conductive needle in a mixture of HCl and HNO₃ with a weight ratio of (8:1) of (HCl:HNO₃), heating the mixture of HCl and HNO₃ containing the second electrically conductive needle to a temperature of 60° C., maintaining the mixture of HCl and HNO₃ containing the second electrically conductive needle at 60° C. for 180 seconds, removing the second electrically conductive needle from the mixture of HCl and HNO₃, washing the second electrically conductive needle with deionized water, and drying the second electrically conductive needle with compressed air.

In an exemplary implementation, generating the nanoporous surface on the outer surface of the second electrically conductive needle may include dry etching of the outer surface of the second electrically conductive needle. In an exemplary implementation, dry etching of the outer surface of the second electrically conductive needle may include placing the second electrically conductive needle in a Reactive Ion Etching (RIE) system and RIE processing of the second electrically conductive needle with a processing mixture comprising SF₆ with a flow rate of 150 sccm and O₂ with a flow rate of 150 sccm for 20 minutes at a radiofrequency (RF) power of 250 W and a processing pressure of 20 mTorr.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a schematic view of an exemplary electrical probe, consistent with one or more exemplary embodiments of the present disclosure.

FIG. 1B illustrates a schematic exploded view of an exemplary electrical probe, consistent with one or more exemplary embodiments of the present disclosure.

FIG. 1C illustrates another schematic exploded view of an exemplary electrical probe, consistent with one or more exemplary embodiments of the present disclosure.

FIG. 2 illustrates an exemplary system for destroying an exemplary cancerous tumor while monitoring electrical impedance variations within the cancerous tumor, consistent with one or more exemplary embodiments of the present disclosure.

FIG. 3A illustrates an exemplary method for destroying an exemplary cancerous tumor, consistent with one or more exemplary embodiments of the present disclosure.

FIG. 3B illustrates an exemplary implementation of an exemplary method for preparing an exemplary electrical probe, consistent with one or more exemplary embodiments of the present disclosure.

FIG. 4 illustrates an exemplary implementation of a model test for calibration of impedimetric properties of normal and cancerous tissues by scanning from 1 Hz to 1 MHz, including an exemplary electrical impedance plot diagram and an exemplary electrical impedance phase plot diagram, consistent with one or more exemplary embodiments of the present disclosure.

FIG. 5 illustrates an example computer system in which an embodiment of the present disclosure, or portions thereof, may be implemented as computer-readable code, consistent with one or more exemplary embodiments of the present disclosure.

FIG. 6A illustrates various parameters for electrochemically production of porous texture on exemplary Pt needles, field emission-scanning electron microscopy (FE-SEM) and bubble production results for different fabricated nanoporous Pt needles at different conditions, consistent with one or more exemplary embodiments of the present disclosure.

FIG. 6B illustrates EDS spectra of an exemplary porous Pt needle fabricated with optimized parameters, including: RF power=250 W, processing mixture=SF₆ (150 sccm)+O₂ (150 sccm), processing time=15 minutes, and processing pressure=20 mTorr, consistent with one or more exemplary embodiments of the present disclosure.

FIG. 7 illustrates FE-SEM images of an exemplary nanoporous Pt needle, consistent with one or more exemplary embodiments of the present disclosure.

FIG. 8 illustrates AFM topographies of an exemplary bare Pt needle and an exemplary nanoporous Pt needle, consistent with one or more exemplary embodiments of the present disclosure.

FIG. 9 illustrates generation of microbubbles in saline by an exemplary bare Pt needle and an exemplary nanoporous Pt needle at about 4 volts, consistent with one or more exemplary embodiments of the present disclosure.

FIG. 10 illustrates ultrasonography images of a treated mouse utilizing an exemplary probe with nanoporous Pt anode electrode, including tumor mass before, tumor cite during, and tumor cite after treatment, consistent with one or more exemplary embodiments of the present disclosure.

FIG. 11 illustrates histological patterns of treated mouse's body organs, including colon, kidney, liver, lung, and spleen, consistent with one or more exemplary embodiments of the present disclosure.

FIG. 12 illustrates histological patterns of Hematoxylin and eosin (H&E) assay for treated mass area utilizing an exemplary non-porous Pt anode electrode and an exemplary porous Pt anode electrode, consistent with one or more exemplary embodiments of the present disclosure.

FIG. 13A illustrates average of active tumor volume due to the radiologist diagnosis (opinion) of treated groups from the first treatment monitored by ultrasonography imaging, consistent with one or more exemplary embodiments of the present disclosure.

FIG. 13B illustrates differences in mean tumor size between treatment with non-porous/porous Pt anode electrode and control groups (TG1 (Non-porous Pt) and TG1 (Porous Pt) versus CG1: p<0.0013 and p<0.0011, TG2 (Non-porous Pt) and TG2 (Porous Pt) versus CG2: p<0.0087 and p<0.0074, and TG3 (Non-porous Pt) and TG3 (Porous Pt) versus CG3: p<0.0096 and p<0.0092) up to twenty days after the first treatment for each group, consistent with one or more exemplary embodiments of the present disclosure.

FIG. 14A illustrates Kaplan-Meier survival curves of 3 groups of treatments (n=10 mice per group), consistent with one or more exemplary embodiments of the present disclosure.

FIG. 14B illustrates Kaplan-Meier survival curves of group 2 (G2) treated mice with porous Pt anode electrode and withdrawn from treatment (n=3 mice per group), consistent with one or more exemplary embodiments of the present disclosure.

FIG. 15A illustrates impedance magnitude and phase diagram for a rat treated by EChT with a bare Pt anode electrode, consistent with one or more exemplary embodiments of the present disclosure.

FIG. 15B illustrates impedance magnitude and phase diagram for a rat treated by EChT with a porous Pt anode electrode, consistent with one or more exemplary embodiments of the present disclosure.

FIG. 16 illustrates ultrasonography images of a tumor site of a treated rat by EChT utilizing an electrical probe with porous Pt anode electrode, including before treatment, the electrical probe placed inside the tumor, about 2 minutes after applying DC, about 15 minutes after starting treatment, and immediately after treatment (25 min), consistent with one or more exemplary embodiments of the present disclosure.

FIG. 17 illustrates ultrasonography images of tumor cite before treatment, EChT treated tumor cite after about five days of first treatment, and tumor cite about five days after second treatment with EChT, consistent with one or more exemplary embodiments of the present disclosure.

FIG. 18 illustrates ultrasonography images of Sarcoma tumor of an exemplary patient 1 before treatment and divided exemplary tumor into two exemplary parts of lateral part and medial part after three cycles of impedance monitoring-assisted EChT treatment, consistent with one or more exemplary embodiments of the present disclosure.

FIG. 19 illustrates decreasing trend of tumor volume for exemplary lateral part and exemplary medial part, consistent with one or more exemplary embodiments of the present disclosure.

FIG. 20 illustrates ultrasonography images of an invasive ductal carcinoma (DC) tumor of an exemplary patient 2 before treatment, consistent with one or more exemplary embodiments of the present disclosure.

FIG. 21 illustrates decreasing trend of tumor volume for exemplary treated IDC tumor of patient 2, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein, an exemplary probe, system, and method is disclosed for in-vivo Single Needle Electrochemical Therapy (SNEChT) of a cancerous tumor. An exemplary probe may include two needle electrodes, where one exemplary electrode may be placed inside another exemplary electrode; allowing for more precise and less invasive Electrochemical Therapy (EChT). An exemplary inner electrode may be movable inside an exemplary outer electrode; allowing for tunable in-situ inner electrode size depending on width and location of an exemplary tumor. Tunable inner electrode size may prevent from over-acidification of treated or non-desired lesions. Hence, tissue over distribution may be reduced. One exemplary electrode may include a nano-porous platinum electrode that may increase an interactive surface between an exemplary probe and a tumor, and, therefore, may produce a higher amount of electrical current with lower stimulating direct current (DC) voltage in comparison with a bare electrode.

An exemplary probe with such characteristics also may be utilized for measuring an efficacy of EChT based on intra-therapeutic impedance recording utilizing the same probe. An exemplary method may include successive steps of EChT and electrical impedance measuring of a tumoral tissue. Monitoring of electrical impedance during EChT may provide insight to a medical expert about local destruction of a tumor in a respective location. So, an expert may be informed about a threshold of tissue acidosis with a lowest electrical stimulation. Briefly, insertion of one needle having a tunable electrode length with a nano-textured surface applied for both precisely EChT and intra-therapeutically electrical impedance monitoring may solve current difficulties of conventional EChT approaches.

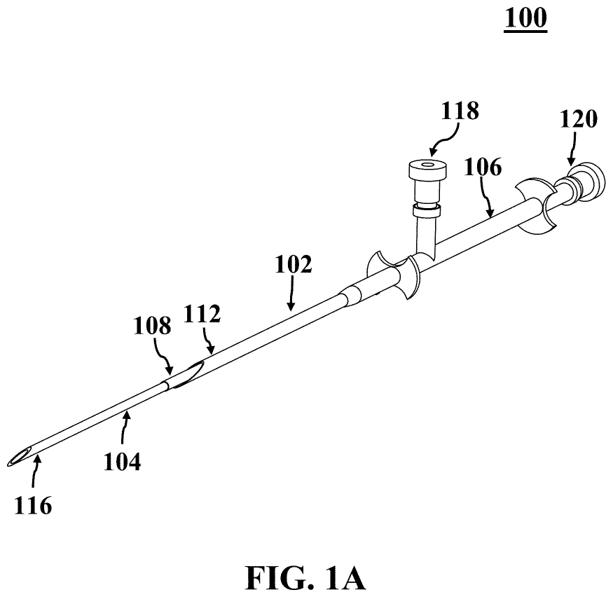

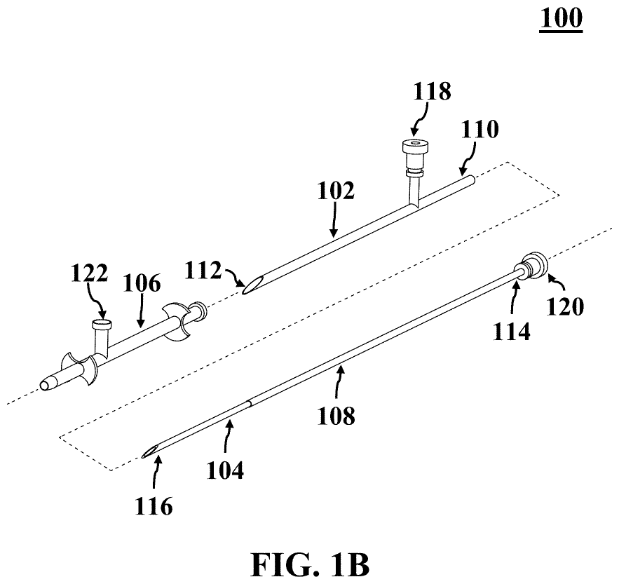

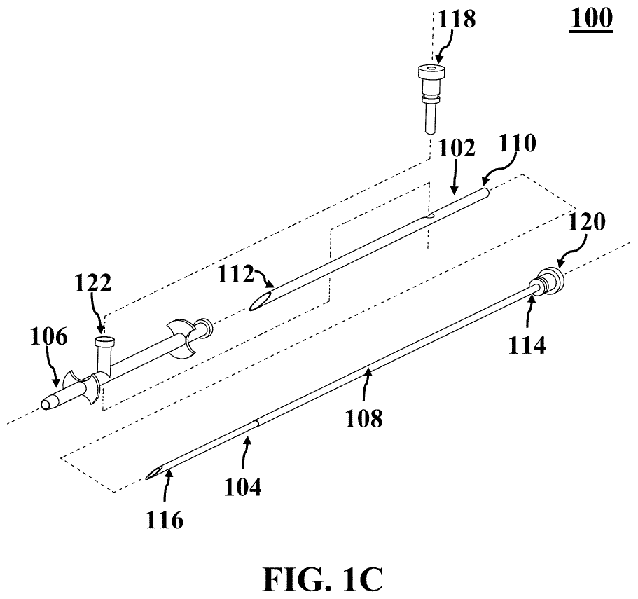

FIG. 1A shows a schematic view of exemplary electrical probe 100, consistent with one or more exemplary embodiments of the present disclosure. Furthermore, FIG. 1B illustrates a schematic exploded view of exemplary electrical probe 100, consistent with one or more exemplary embodiments of the present disclosure. Additionally, FIG. 1C illustrates another schematic exploded view of exemplary electrical probe 100, consistent with one or more exemplary embodiments of the present disclosure. In an exemplary embodiment, electrical probe 100 may include a first electrode 102 and a second electrode 104. In an exemplary embodiment, the second electrode 104 may be placed inside the first electrode 102.

In an exemplary embodiment, the first electrode 102 and the second electrode 104 may comprise a couple of an exemplary cathode electrode and an exemplary anode electrode for utilizing electrical probe 100 in electrochemical applications, such as an EChT process. In an exemplary embodiment, the first electrode 102 may be utilized as an exemplary cathode electrode and the second electrode 104 may be utilized as an exemplary anode electrode. In another exemplary embodiment, the first electrode 102 may be utilized as an exemplary anode electrode and the second electrode 104 may be utilized as an exemplary cathode electrode. In an exemplary embodiment, the first electrode 102 and the second electrode 104 may comprise a couple of electrical electrodes for electrical measurement applications, such as electrical impedance measurement of a sample.

In an exemplary embodiment, the first electrode 102 may include a first electrically conductive needle. In an exemplary embodiment, the first electrode 102 may include a needle made of stainless steel. In an exemplary embodiment, the first electrode 102 may include a hollow needle; allowing for placing second electrode 104 there inside. In an exemplary embodiment, the first electrode 102 may include a needle of a peripheral venous catheter. In an exemplary embodiment, the first electrode 102 may include a needle of a peripheral venous catheter with a gauge size of gauge 14 or more, for example, at least one of gauge 16, gauge 18, and gauge 20 and so on. In an exemplary embodiment, the first electrode 102 may include a needle of a peripheral venous catheter with an external diameter of about 2.1 mm or less. In an exemplary embodiment, the first electrode 102 may include a needle of a peripheral venous catheter with a size of gauge 14 that may have an external diameter of about 2.1 mm and a length of about 45 mm.

In an exemplary embodiment, the second electrode 104 may include a second electrically conductive needle. In an exemplary embodiment, the second electrically conductive needle may have an external diameter less than an internal diameter of the first electrically conductive needle. In an exemplary embodiment, the second electrically conductive needle may include a hollow needle. In an exemplary embodiment, the second electrode 104 may be placed inside the first electrode 102. In an exemplary embodiment, the second electrode 104 may be movable in a longitudinal direction along the first electrode 102 while being inside the first electrode 102.

In an exemplary embodiment, the second electrode 104 may include a needle made of at least one of stainless steel and platinum-iridium alloy. In an exemplary embodiment, the second electrode 104 may include a needle made of platinum-iridium alloy with a weight ratio of 9:1 for platinum: iridium. In an exemplary embodiment, the second electrode 104 may have a thickness between about 100 μm and about 500 μm.

In an exemplary embodiment, an outer surface of the second electrode 104 may include a nanoporous surface. In an exemplary embodiment, the nanoporous surface of the second electrode 104 may include an electrically etched outer surface of the second electrically conductive needle. In an exemplary embodiment, the electrically etched outer surface may include a plurality of nanopores with a diameter of less than about 500 nanometers. In an exemplary embodiment, the electrically etched outer surface may include a plurality of nanopores with a diameter of less than about 300 nanometers. In an exemplary embodiment, the electrically etched outer surface may include a plurality of nanopores with a diameter of less than about 100 nanometers. In an exemplary embodiment, the nanoporous surface of the second electrode 104 may include a plurality of nano-sized hills on an outer surface of the second electrically conductive needle with a width of less than 100 nm and a depth of less than 100 nm for each respective hill of the plurality of nano-sized hills. In an exemplary embodiment, smaller and more uniform sizes of the plurality of nanopores may lead to a more effective electrolysis of a cancerous tumor in contact with the first electrode 102 and the second electrode 104 in an exemplary EChT treatment of the cancerous tumor. Additionally, the smaller and more uniform sizes of the plurality of nanopores may further lead to more accurate electrical measurements utilizing electrical probe 100, such as impedance measurements.

In an exemplary embodiment, the first electrode 102 may include a first distal end portion 112 and a first proximal end portion 110. In an exemplary embodiment, the second electrode 104 may include a second distal end portion 116 and a second proximal end portion 114. In an exemplary embodiment, the first distal end portion 112 and the second distal end portion 116 may define (comprise) a respective length of the first electrode 102 and the second electrode 104 to be inserted into a sample or to be put in contact with the sample. In an exemplary embodiment, the first distal end portion 112 and the second distal end portion 116 may be configured to be put in contact with a biological sample. In an exemplary embodiment, the biological sample may include a portion of a cancerous tumor in a living tissue. In an exemplary embodiment, the first distal end portion 112 and the second distal end portion 116 may be configured to be inserted into the portion of the cancerous tumor. In an exemplary embodiment, both of the first electrode 102 and the second electrode 104 may have sharp pointed tips at their receptive distal ends 112 and 116; leading to simply and less-invasive insertion of the first electrode 102 and the second electrode 104 into a sample.

In an exemplary embodiment, the second distal end portion 116 may be placed inside or outside of the first electrode 102. In an exemplary embodiment, the second distal end portion 116 may be placed (adjusted to be placed) outside of the first electrode 102 while putting the first distal end portion 112 and the second distal end portion 116 in contact with the biological sample. In an exemplary embodiment, a length of the second distal end portion 116 may be tunable by moving the second electrode 104 in longitudinal direction inside the first electrode 102 depending on a desired depth of a sample to be put in contact with the second electrode 104. In an exemplary embodiment, the length of the second distal end portion 116 may be tunable by pushing and/or pulling the second electrode 104 in longitudinal direction inside the first electrode 102 depending on the desired depth of the sample to be put in contact with the second electrode 104. In an exemplary embodiment, a length of the second distal end portion 116 that may be placed outside of the first electrode 102 may be adjusted based on a depth of a target location of a cancerous tumor to be put in contact with the first electrode 102 and the second electrode 104. In an exemplary embodiment, the length of the second distal end portion 116 that may be placed outside of the first electrode 102 may be adjusted by pushing and/or pulling the second electrode 104 in longitudinal direction inside the first electrode 102.

In an exemplary embodiment, electrical probe 100 may further include two respective electrically insulating layers 106 and 108 coated on respective outer surfaces of the first electrode 102 and the second electrode 104. In an exemplary embodiment, electrically insulating layers 106 and 108 may be partially coated on respective outer surfaces of the first electrode 102 and the second electrode 104 so that the first distal end portion 112 and the second distal end portion 116 may be remained uncoated. In an exemplary embodiment, electrically insulating layers 106 and 108 may be configured to prevent electrolysis of non-targeted internal organs and skin during applying a direct current (DC) voltage to the first electrode 102 and the second electrode 104 in an exemplary EChT process. In an exemplary embodiment, electrically insulating layers 106 and 108 may prevent transferring electricity to non-targeted internal organs and skin throughout a length of an insertion path of the first electrode 102 and the second electrode 104 within a patient's body; thereby, result in preventing electrolysis of the non-targeted internal organs and skin. Furthermore, electrically insulating layers 106 and 108 may prevent direct contact of the first electrode 102 and the second electrode 104 with each other; thereby, resulting in preventing electrical noises in an exemplary EChT process or an exemplary electrical impedance measurement. In an exemplary embodiment, electrically insulating layers 106 and 108 may include first electrically insulating layer 106 coated around the first electrode 102 except the first distal end portion 112 and second electrically insulating layer 108 coated around the second electrode 104 except the second distal end portion 116; allowing for insulating portions of a sample or a patient's body adjacent to the cancerous tumor from electrical signals that may be applied to electrical probe 100.

In an exemplary embodiment, the first electrically insulating layer 106 may include a tubular cover placed around the first electrode 102 except the first distal end portion 112. In an exemplary embodiment, the first electrically insulating layer 106 may include a plastic cannula covered the first electrode 102 except the first distal end portion 112. In an exemplary embodiment, the first electrically insulating layer 106 may include a layer of an electrically insulating material coated around the first electrode 102 except the first distal end portion 112; allowing for putting the first distal end portion 112 of the first electrode 102 in contact with a sample. The first electrically insulating layer 106 may be configured to protect surrounding tissue(s) of an exemplary cancerous tumor from being in contact with the first electrode 102 to prevent possible side effects of an electrical signal applied to and/or received from electrical probe 100, that is, due to the fact that the first electrically insulating layer 106 may include an electrically insulating material, surround tissue of an exemplary cancerous tumor may be insulated from first electrode 102.

In an exemplary embodiment, the second electrically insulating layer 108 may include a layer of an electrically insulating material that may be placed between the first electrode 102 and the second electrode 104. In an exemplary embodiment, the second electrically insulating layer 108 may include a layer of an electrically insulating material coated around the second electrode 104 except the second distal end portion 116. The second distal end portion 116 may be kept uncoated as an electrically interactive length of the second electrode 104 that may be configured to be put in contact with an exemplary cancerous tumor. The second electrically insulating layer 108 may be configured to be electrically insulating the first electrode 102 and the second electrode 104 from each other in order to prevent possible electrical noises while applying an electrical signal to electrical probe 100 and/or receiving an electrical signal from electrical probe 100. Furthermore, the second electrically insulating layer 108 may be configured to be electrically insulating electrically insulating surrounding tissue(s) of an exemplary cancerous tumor from being in contact with the second electrode 104 in order to prevent possible side effects of an electrical signal applied to and/or received from electrical probe 100.

Referring to FIGS. 1A-1C, electrical probe 100 may further include two electrical connectors 118 and 120. In an exemplary embodiment, first electrical connector 118 may be attached onto a surface of the first electrode 102 adjacent to the first proximal end portion 110 of the first electrode 102. In an exemplary embodiment, the first electrically insulating layer 106 may include exemplary opening 122 adjacent to the first proximal end portion 110. In an exemplary embodiment, the first electrical connector 118 may be passed through opening 122 and attached onto the surface of first electrode 102. In an exemplary embodiment, the first electrical connector 118 may be configured to connect the first electrode 102 to an exemplary electrical device via connecting the first electrical connector 118 to the electrical device. In an exemplary embodiment, the first electrical connector 118 may be configured to be connected to at least one of an impedance analyzer device and a DC voltage generator.

In an exemplary embodiment, second electrical connector 120 may be attached onto a surface of the second electrode 104 adjacent to the second proximal end portion 114 of the second electrode 104. In an exemplary embodiment, the second electrical connector 120 may be configured to connect the second electrode 104 to an exemplary electrical device via connecting second electrical connector 120 to the electrical device. In an exemplary embodiment, second electrical connector 120 may be configured to be connected to at least one of an impedance analyzer device and a DC voltage generator.

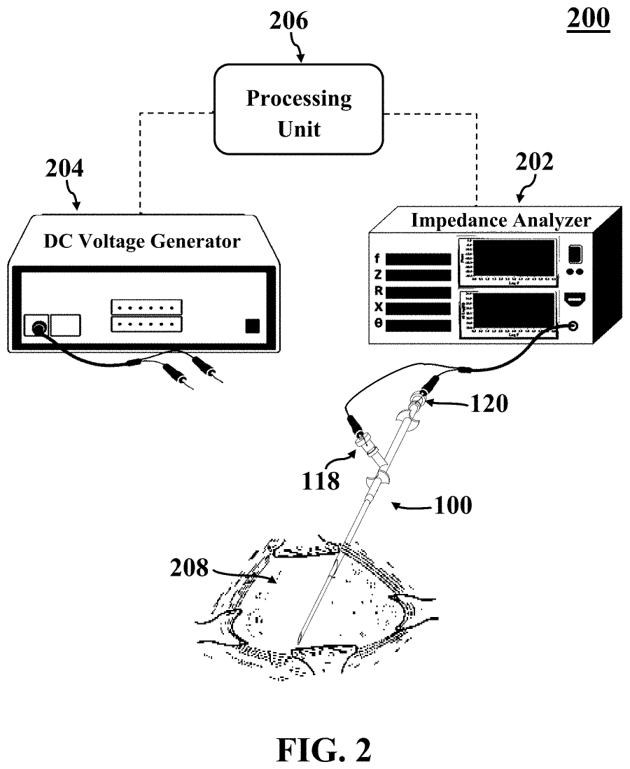

In an exemplary implementation of the present disclosure, exemplary electrical probe 100 may be utilized via an exemplary method and an exemplary system for destroying an exemplary cancerous tumor and/or monitoring electrical impedance of an exemplary cancerous tumor. In an exemplary embodiment, electrical probe 100 may be utilized via an exemplary method in an exemplary system for destroying an exemplary cancerous tumor via electrochemical electrolysis of peripheral medium of cancer cells within the cancerous tumor. The system and method may be further utilized for monitoring variations of electrical impedance of the cancerous tumor during destruction of cancer cells. FIG. 2 shows exemplary system 200 for destroying exemplary cancerous tumor 208 while monitoring electrical impedance variations within the cancerous tumor, consistent with one or more exemplary embodiments of the present disclosure. In an exemplary embodiment, exemplary system 200 may include electrical probe 100, impedance analyzer device 202, DC voltage generator 204, and processing unit 206. In an exemplary embodiment, impedance analyzer device 202 and DC voltage generator 204 may be configured to be connected to electrical probe 100. In an exemplary embodiment, processing unit 206 may be electrically connected to impedance analyzer device 202 and DC voltage generator 204.

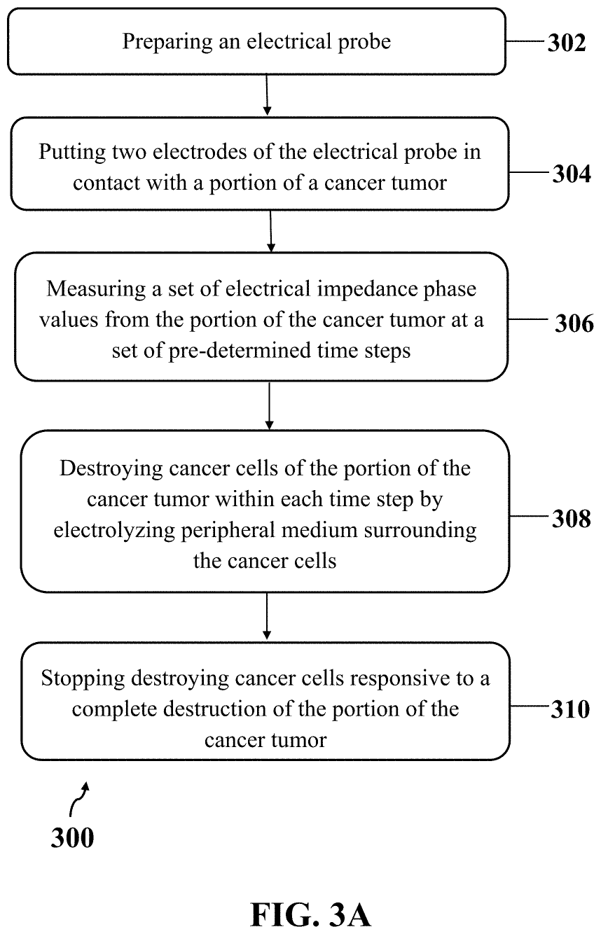

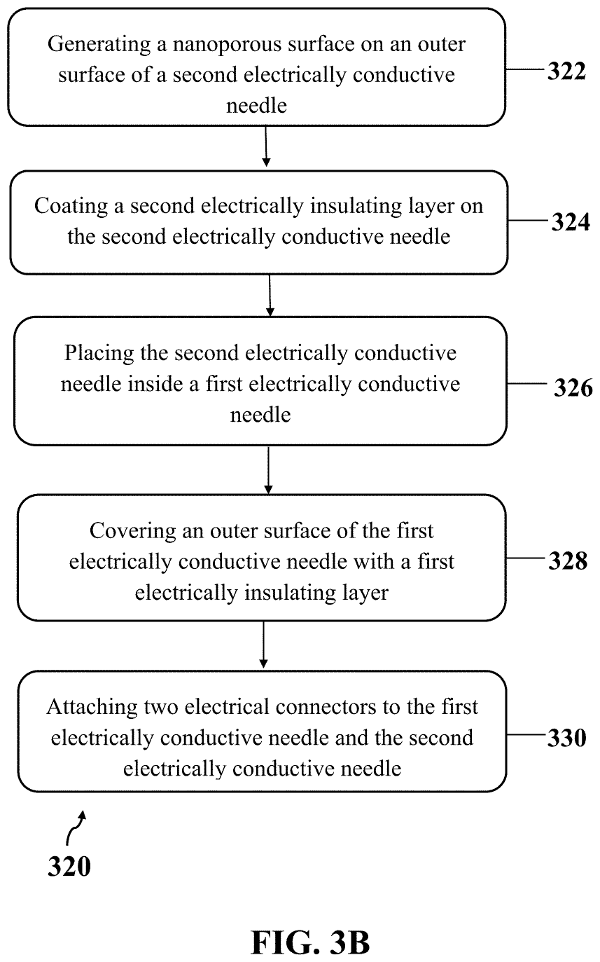

In an exemplary implementation, exemplary electrical probe 100 and exemplary system 200 may be utilized via an exemplary method for destroying an exemplary cancerous tumor and/or monitoring electrical impedance of an exemplary cancerous tumor. FIG. 3A shows exemplary method 300 for destroying exemplary cancerous tumor 208, consistent with one or more exemplary embodiments of the present disclosure. In an exemplary implementation, method 300 may include preparing electrical probe 100 (step 302), putting two electrodes of electrical probe 100 in contact with a portion of cancerous tumor 208 (step 304), plotting an impedance phase diagram by measuring a set of electrical impedance phase values from the portion of cancerous tumor 208 at a respective set of pre-determined time steps (step 306), destroying cancer cells of the portion of cancerous tumor 208 within each time step by electrolyzing peripheral medium surrounding the cancer cells of the portion of cancerous tumor 208 (step 308), and stopping destroying of the cancer cells responsive to a complete destruction of the portion of cancerous tumor 208 (step 310). In an exemplary implementation, cancerous tumor 208 may include all types of cancerous tumors in animals or human's body. In an exemplary implementation, cancerous tumor 208 may include a breast tumor, a malignant breast tumor, an invasive ductal carcinoma (IDC) tumor, a soft tissue sarcoma tumor, such as Leiomyosarcoma and Spindle cell sarcoma, etc.

In detail, step 302 may include preparing electrical probe 100. FIG. 3B shows an exemplary implementation of exemplary method 320 for preparing electrical probe 100 (step 302), consistent with one or more exemplary embodiments of the present disclosure. In an exemplary implementation, method 320 for preparing electrical probe 100 may include forming the second electrode 104 by generating a nanoporous surface on an outer surface of the second electrically conductive needle (step 322), coating the second electrically insulating layer 108 on the second electrically conductive needle except the second distal end portion 116 of the second electrically conductive needle (step 324), placing the second electrically conductive needle inside the first electrically conductive needle (step 326), covering an outer surface of the first electrically conductive needle with the first electrically insulating layer 106 except the first distal end portion 112 of the first electrically conductive needle (step 328), and attaching two electrical connectors 118 and 120 to the first electrically conductive needle and the second electrically conductive needle (step 330).

In an exemplary implementation, generating the nanoporous surface on the outer surface of the second electrically conductive needle (step 322) may include forming a plurality of nano-features on the outer surface of the second electrically conductive needle. In an exemplary implementation, forming the plurality of nano-features on the outer surface of the second electrically conductive needle may include forming a plurality of nano-sized hills on the outer surface of the second electrically conductive needle with a width of less than 100 nm and a depth of less than 100 nm for each respective hill of the plurality of nano-sized hills.

In an exemplary implementation, generating the nanoporous surface on the outer surface of the second electrically conductive needle (step 322) may include forming a plurality of nanopores with a diameter of less than about 500 nanometers. In an exemplary embodiment, each nanopore of the plurality of nanopores may have a diameter of less than about 300 nanometers. In another exemplary embodiment, the plurality of nanopores may have a diameter of less than about 100 nanometers.

In an exemplary implementation, generating the nanoporous surface on the outer surface of the second electrically conductive needle (step 322) may include at least one of wet etching of the outer surface of the second electrically conductive needle and dry etching of the outer surface of the second electrically conductive needle. In an exemplary implementation, wet etching of the outer surface of the second electrically conductive needle may include placing the second electrically conductive needle in a mixture of HCl and HNO₃ with a weight ratio of about 8:1 of HCl:HNO₃, heating the mixture of HCl and HNO₃ containing the second electrically conductive needle to a temperature of about 60° C., maintaining the mixture of HCl and HNO₃ containing the second electrically conductive needle at about 60° C. for about 180 seconds, removing the second electrically conductive needle from the mixture of HCl and HNO₃, washing the second electrically conductive needle with deionized water, and drying the second electrically conductive needle with compressed air.

In an exemplary implementation, dry etching of the outer surface of the second electrically conductive needle may include placing the second electrically conductive needle in a Reactive Ion Etching (RIE) system and RIE processing of the second electrically conductive needle. In an exemplary implementation, applied conditions for RIE processing of the second electrically conductive needle may be changed to obtain a desired nanoporous surface on the outer surface of the second electrically conductive needle with a high and homogenous nanoporous surface. In an exemplary implementation, applied conditions for RIE processing of the second electrically conductive needle may include utilizing a processing mixture including SF₆ with a flow rate of about 150 sccm and O₂ with a flow rate of about 150 sccm for about 20 minutes at a radiofrequency (RF) power of about 250 W and a processing pressure of about 20 mTorr.

In an exemplary implementation, step 324 may include coating the second electrically insulating layer 108 on the second electrically conductive needle except the second distal end portion 116 of the second electrically conductive needle. In an exemplary implementation, coating the second electrically insulating layer 108 on the second electrically conductive needle may include adhering a thin layer of biocompatible plastic cannula on the second electrically conductive needle except the second distal end portion 116 of the second electrically conductive needle. In an exemplary implementation, coating the second electrically insulating layer 108 on the second electrically conductive needle may include adhering the thin layer of biocompatible plastic cannula on the second electrically conductive needle and peeling a length of the adhered biocompatible plastic cannula on the second distal end portion 116 of the second electrically conductive needle. In an exemplary implementation, adhering the thin layer of biocompatible plastic cannula on the second electrically conductive needle may be done by heating the thin layer of biocompatible plastic cannula and/or the second electrically conductive needle. In an exemplary implementation, adhering the thin layer of biocompatible plastic cannula on the second electrically conductive needle may include adhering a hot thin layer of biocompatible plastic cannula on the second electrically conductive needle.

In an exemplary implementation, attaching two electrical connectors 118 and 120 to the first electrically conductive needle and the second electrically conductive needle (step 330) may include attaching the first electrical connector 118 onto a surface of the first electrically conductive needle adjacent to the first proximal end portion 110 of the first electrically conductive needle and attaching the second electrical connector 120 onto the second proximal end portion 114 of the second electrically conductive needle.

Referring again to FIG. 3A, step 304 may include putting two electrodes 102 and 104 of electrical probe 100 in contact with a portion of cancerous tumor 208 with reference to FIG. 2. In an exemplary implementation, putting two electrodes 102 and 104 of electrical probe 100 in contact with an exemplary portion of cancerous tumor 208 may include putting the first electrode 102 and the second electrode 104 of electrical probe 100 in contact with an exemplary portion of cancerous tumor 208. In an exemplary implementation, putting two electrodes 102 and 104 of electrical probe 100 in contact with an exemplary portion of cancerous tumor 208 may include inserting the first electrode 102 and the second electrode 104 of electrical probe 100 into an exemplary portion of cancerous tumor 208. An exemplary portion of cancerous tumor 208 may refer to a portion of cancerous tumor 208 that is to be targeted.

In an exemplary implementation, putting two electrodes 102 and 104 of electrical probe 100 in contact with an exemplary portion of cancerous tumor 208 may include putting the first distal end portion 112 of the first electrode 102 and the second distal end portion 116 of the second electrode 104 of electrical probe 100 in contact with an exemplary portion of cancerous tumor 208. In an exemplary implementation, putting two electrodes 102 and 104 of electrical probe 100 in contact with an exemplary portion of cancerous tumor 208 may include inserting the first distal end portion 112 of the first electrode 102 into an exemplary portion of cancerous tumor 208 and pushing the second distal end portion 116 of the second electrode 104 through the first electrode 102 into an exemplary portion of cancerous tumor 208.

In an exemplary implementation, putting two electrodes 102 and 104 of electrical probe 100 in contact with an exemplary portion of cancerous tumor 208 may further include identifying a location of an exemplary portion of cancerous tumor 208; allowing for applying steps of method 300 to an accurate location of a cancerous tumor and not a non-cancerous region, for example, a lipid region. In an exemplary implementation, putting two electrodes 102 and 104 of electrical probe 100 in contact with an exemplary portion of cancerous tumor 208 may further include identifying the location of an exemplary portion of cancerous tumor 208 by at least one of monitoring putting two electrodes 102 and 104 of electrical probe 100 in contact with an exemplary portion of cancerous tumor 208 with guide of sonography imaging, comparing an electrical impedance value of a location in contact with two electrodes 102 and 104 of electrical probe 100 with a reference impedance value, and combinations thereof. In an exemplary implementation, identifying the location of an exemplary portion of cancerous tumor 208 may be done by at least one of monitoring inserting the first distal end portion 112 of the first electrode 102 of electrical probe 100 into an exemplary portion of cancerous tumor 208 with guide of sonography imaging, comparing an electrical impedance value of a location of the inserted the first distal end portion 112 of the first electrode 102 of electrical probe 100 with a reference impedance value, and combinations thereof.

In an exemplary implementation, identifying the location of an exemplary portion of cancerous tumor 208 by comparing the electrical impedance value of the location of the inserted the first distal end portion 112 of the first electrode 102 with the reference impedance value may include connecting two electrodes 102 and 104 of electrical probe 100 to impedance analyzer device 202, applying an AC voltage in a sweeping range of frequencies between about 1 Hz and about 1 MHz to two electrodes 102 and 104, measuring an electrical impedance value of the location of the inserted the first distal end portion 112 of the first electrode 102 at a reference frequency value, and identifying location of the inserted the first distal end portion 112 of the first electrode 102 as the location of an exemplary portion of cancerous tumor 208 if the measured electrical impedance value is less than the reference impedance value. In an exemplary implementation, identifying a location of an exemplary portion of cancerous tumor 208 by comparing the electrical impedance value of a location of the inserted the first distal end portion 112 of the first electrode 102 with a reference impedance value may include connecting two electrodes 102 and 104 of electrical probe 100 to impedance analyzer device 202, applying an AC voltage with the reference frequency value to two electrodes 102 and 104, measuring an electrical impedance value of a location of the inserted the first distal end portion 112 of the first electrode 102 at the reference frequency value, and identifying a location of the inserted the first distal end portion 112 of the first electrode 102 as the location of the portion of cancerous tumor 208 if the measured electrical impedance value is less than the reference impedance value. In an exemplary embodiment, the reference frequency value may include a frequency value of about 1 kHz and the reference impedance value may include an impedance value of about 1500Ω.

In an exemplary implementation, putting two electrodes of electrical probe 100 in contact with an exemplary portion of cancerous tumor 208 may be carried out while conducting sonography imaging; allowing for precise insertion of the first electrode 102 and the second electrode 104 into an exemplary portion of cancerous tumor 208.

Moreover, referring to FIG. 3A, step 306 may include plotting an impedance phase diagram by measuring a set of electrical impedance phase values from an exemplary portion of cancerous tumor 208 at a respective set of pre-determined time steps. In an exemplary implementation, measuring the set of electrical impedance phase values from an exemplary portion of cancerous tumor 208 at the respective set of pre-determined time steps may include measuring a set of electrical impedance phase values from an exemplary portion of cancerous tumor 208 at end of each time step and between each two successive time steps of the set of pre-determined time steps. In an exemplary implementation, measuring the set of electrical impedance phase values from an exemplary portion of cancerous tumor 208 at the respective set of pre-determined time steps may be done by applying an alternating current (AC) voltage in a sweeping range of frequencies to the first electrode 102 and the second electrode 104 between each two successive time steps of the set of pre-determined time steps utilizing impedance analyzer device 202 connected to electrical probe 100 via the first electrical connector 118 and the second electrical connector 120. Furthermore, measuring the set of electrical impedance phase values from an exemplary portion of cancerous tumor 208 may further include measuring an initial electrical impedance phase value before starting a first time step of the set of pre-determined time steps.

In an exemplary implementation, the set of pre-determined time steps may include at least one of a set of equal time steps, a set of varying (unequal) time steps, and combinations thereof. In an exemplary implementation, the set of pre-determined time steps may be passed in series sequentially after each other during applying exemplary method 300. In an exemplary implementation, each time step of the set of pre-determined time steps may include a time period in a range between about 1 second and about 5 minutes. In an exemplary implementation, each time step may include a time period in a range between about 5 seconds and about 3 minutes. In an exemplary implementation, each time step may include a time period in a range between about 1 minutes and about 3 minutes. In an exemplary implementation, each time step may include a time period of about 15 seconds.

In an exemplary implementation, measuring each electrical impedance phase value of the set of electrical impedance values at end of each time step of the set of pre-determined time steps from an exemplary portion of cancerous tumor 208 may include connecting the first electrode 102 and the second electrode 104 of electrical probe 100 to impedance analyzer device 202, applying an AC voltage in a sweeping range of frequencies between about 1 Hz and about 1 MHz to the first electrode 102 and the second electrode 104, measuring the set of electrical impedance phase values respective to the swept range of frequencies, and plotting the measured set of electrical impedance phase values versus the swept range of frequencies. In an exemplary implementation, measuring each electrical impedance phase value of the set of electrical impedance values from an exemplary portion of cancerous tumor 208 may be done during a time period in a range between about 5 second and about 30 seconds at the end of each time step of the set of pre-determined time steps.

In an exemplary implementation, connecting the first electrode 102 and the second electrode 104 of electrical probe 100 to impedance analyzer device 202 may include connecting the first electrical connector 118 and the second electrical connector 120 to impedance analyzer device 202. In an exemplary implementation, applying the AC voltage in the sweeping range of frequencies may include applying the AC voltage in a sweeping range of frequencies between about 100 kHz and about 500 kHz to the first electrode 102 and the second electrode 104.

In an exemplary implementation, plotting the impedance phase diagram in step 306 and identifying a location of an exemplary portion of cancerous tumor 208 in step 304 may be done via an electrochemical impedance spectroscopy (EIS) approach. The EIS approach may include applying a known voltage or current as an electrical stimulus to a biological material (e.g., a location within an animal's or a person's body where the first electrode 102 and the second electrode 104 may be inserted there) and measuring a resulting current or voltage as a response. In an exemplary embodiment, the first electrode 102 and the second electrode 104 inserted into the location within the animal's or the person's body may be configured to act as impedance stimulation and measurement electrodes. The biological material may produce a complex electrical impedance in response to the electrical stimulus. The complex electrical impedance may depend on the biological material's composition, structures, health status, and physiological or pathological properties. The EIS approach may involve measuring at least one of electrical impedance Z, admittance Y, impedance modulus 14, the permittivity, and combinations thereof as a function of frequency to characterize the biological material. The biological material may conduct an electric current and hence may have an associated impedance parameter. The biological material may include cells and extracellular medium. The cells may be made of cell membrane and intracellular medium. Both extracellular and intracellular medium may include ionic solutions that may be electrically resistive. The cell membrane may be made of a lipid bilayer and proteins and may be primarily capacitive. An electrical impedance associated with this capacitance may be dependent on frequency.

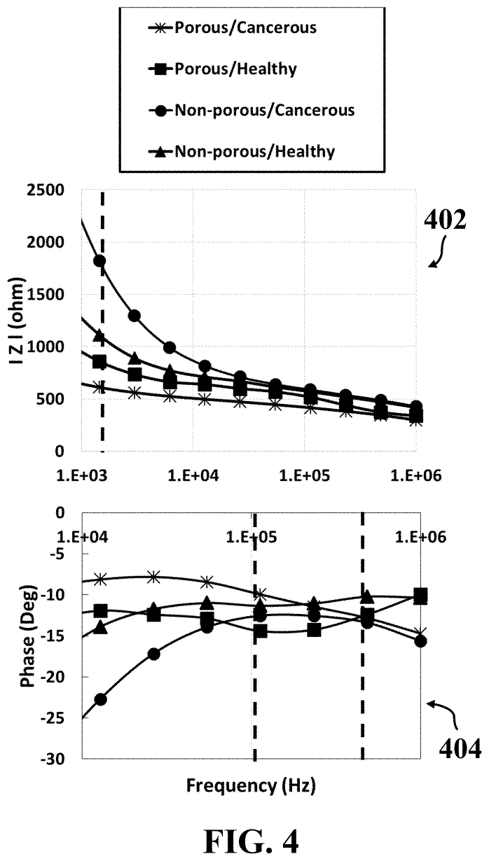

In an exemplary implementation, plotting the impedance phase diagram in step 306 and identifying the location of an exemplary portion of cancerous tumor 208 in step 304 may be done based on comparative responses of the EIS approach for known normal and cancerous tissues in order to calibrate exemplary electrical probe 100 and exemplary method 300. FIG. 4 shows an exemplary implementation of a model test for calibration of impedimetric properties of normal and cancerous tissues by scanning from 1 Hz to 1 MHz, including exemplary electrical impedance plot diagram 402 and exemplary electrical impedance phase plot diagram 404, consistent with one or more exemplary embodiments of the present disclosure. Mice model with normal and cancerous breast tissues utilizing exemplary probe 100 with exemplary non-porous Pt electrode and exemplary porous Pt electrode as the second electrode 104 were used for model test for calibration of impedimetric properties of normal and cancerous tissues. A maximum transmitted power from exemplary probe 100 to tissues may be about 0.8 mW which may be completely in safe mode for a living body. It may be observed from FIG. 4 that there may be a drastic difference in slope of electrical impedance phase diagram (IPS) in a frequency range between about 100 kHz and about 500 kHz for muscular and tumor tissues. It should be noted that diagrams similar to exemplary electrical impedance plot diagram 402 and exemplary electrical impedance phase plot diagram 404 were also obtained for human model tests. Accordingly, the IPS may be the most important parameter in classifying tissues utilizing electrical probe 100 with needle electrodes. As it may be inferred from FIG. 4, normal (healthy) tissue may show a positive IPS while cancerous tissue may show a sharp negative IPS that may be an effective classification parameter. Also, a difference between electrical impedance amplitude and phase in distinguishing between healthy and cancerous tissues may be about 1.5-3.5 times sharper in the porous needle electrodes compared to the non-porous needle electrodes.

In addition, with reference back to FIG. 3A, step 308 may include destroying cancer cells of an exemplary portion of cancerous tumor 208 within each time step (during each time step) by electrolyzing peripheral medium surrounding the cancer cells of an exemplary portion of cancerous tumor 208. In an exemplary implementation, electrolyzing the peripheral medium may include applying a direct current (DC) voltage between the first electrode 102 and the second electrode 104 of electrical probe 100 utilizing DC voltage generator 204 connected to electrical probe 100 via the first electrical connector 118 and the second electrical connector 120.

In an exemplary implementation, electrolyzing peripheral medium surrounding the cancer cells of an exemplary portion of cancerous tumor 208 may include connecting the first electrode 102 and the second electrode 104 of electrical probe 100 to DC voltage generator 204 and applying a DC voltage with a magnitude between about 0.5 V and about 10 V between the first electrode 102 and the second electrode 104 during each time step utilizing DC voltage generator 204. In an exemplary implementation, connecting the first electrode 102 and the second electrode 104 of electrical probe 100 to DC voltage generator 204 may include connecting the first electrical connector 118 and the second electrical connector 120 to DC voltage generator 204.

In an exemplary implementation, electrolyzing peripheral medium surrounding the cancer cells of an exemplary portion of cancerous tumor 208 may result in destroying or necrosis of cancer cell in an exemplary portion of cancerous tumor 208. In an exemplary implementation, electrolyzing peripheral medium surrounding the cancer cells of an exemplary portion of cancerous tumor 208 may produce electrolysis products that may make a region surrounding an anode electrode (e.g., the second electrode 104) very acidic (pH of about 2), and a region surrounding a cathode electrode (e.g., the first electrode 102) strongly alkaline (pH of about 12). At these extreme pH values, tissue's proteins may become denatured and cell structure may collapse, and cell eventually may die. In an exemplary implementation, electrolyzing peripheral medium surrounding the cancer cells of an exemplary portion of cancerous tumor 208 may lead to pH changes and releasing chlorine gas and hydroxyl free radicals; therefore, resulting in a locally cytotoxic microenvironment that may induce coagulative and colliquative necrosis in an area of an exemplary portion of cancerous tumor 208 in contact with the first electrode 102 and the second electrode 104. As a result, coagulation and dehydration may be obtained in acidic media around the anode electrode (e.g., the second electrode 104) as well as colliquative patterns and edema in basic media around the cathode electrode (e.g., the first electrode 102).

In an exemplary implementation, destroying of exemplary cancer cells of an exemplary portion of cancerous tumor 208 within each time step may further include natural degradation of dead cells within a patient's body or animal's body, or natural absorption of dead cells by the patient's body or the animal's body without a further need to remove dead cells from a patient's body or an animal's body. In another exemplary implementation, destroying of exemplary cancer cells of an exemplary portion of cancerous tumor 208 within each time step may further include removing dead cells from a patient's body or an animal's body, for example, by vacuum suction of dead cells.

In an exemplary implementation, destroying of exemplary cancer cells of an exemplary portion of cancerous tumor 208 within each time step may further include vacuum suction of dead cells due to electrolyzing peripheral medium surrounding the cancer cells of an exemplary portion of cancerous tumor 208. In an exemplary implementation, vacuum suction of dead cells may include connecting a vacuum pump to a proximal end of electrical probe 100, and driving out the dead cells from an exemplary portion of cancerous tumor 208 by applying a vacuum pressure more than about 20 KPa to the proximal end of electrical probe 100. In an exemplary implementation, vacuum suction of dead cells may be done utilizing a surgical suction pump with a suction pressure of more than about 20 KPa vacuum pressure corresponding to a suction flow rate of more than about 20 lit/min. In an exemplary implementation, vacuum suction of dead cells may be done utilizing a surgical suction pump with a suction pressure of more than about 60 KPa vacuum pressure or a surgical suction pump with a suction flow rate of about 90 lit/min or more. In an exemplary implementation, connecting a vacuum pump to a proximal end of electrical probe 100 may include connecting a tube of the vacuum pump to a proximal end of electrical probe 100.

In an exemplary implementation, connecting the vacuum pump to a proximal end of electrical probe 100 may include pulling out the second electrode 104 from first electrode 102 and connecting the vacuum pump to first proximal end portion 110 of first electrode 102 via a tube line of the vacuum pump. Furthermore, driving out the dead cells from an exemplary portion of cancerous tumor 208 may be done by applying a vacuum pressure more than about 20 KPa to first proximal end portion 110 of first electrode 102.

Furthermore, step 310 may include stopping destroying of exemplary cancer cells if a complete destruction of an exemplary portion of cancerous tumor 208 is achieved. In an exemplary implementation, stopping destroying of exemplary cancer cells may include ceasing to apply the DC voltage between the first electrode and the second electrode. In an exemplary implementation, stopping destroying of exemplary cancer cells may include calculating the IPS at end of each time step, comparing the calculated IPS with zero, detecting a complete destruction of an exemplary portion of cancerous tumor 208 if the calculated IPS is more than zero or positive, and ceasing to apply the DC voltage between the first electrode 102 and the second electrode 104. In an exemplary implementation, the complete destruction of an exemplary portion of cancerous tumor 208 may include obtaining a positive slope of the impedance phase diagram (IPS) by analyzing the IPS at the end of each time step. In an exemplary implementation, analyzing the IPS at the end of each time step may include calculating the IPS at the end of each time step, comparing the calculated IPS with zero, and detecting the complete destruction of an exemplary portion of cancerous tumor 208 if the calculated IPS is more than zero or positive.

In an exemplary implementation, calculating the IPS at the end of each time step may include calculating the IPS at the end of each time step using Equation (1) as follows:

$$IPS = \frac{{Phase}_{2} - {Phase}_{1}}{log ( {Frequency}_{2} ) - log ( {Frequency}_{1} )} , \qquad Equation ( 1 )$$

In Equation (1), Phase₂ may be a measured impedance phase value at frequency value of Frequency₂ and Phase₁ may be a measured impedance phase value at frequency value of Frequency₁. In an exemplary implementation, Frequenc₁, Frequency₂, Phase₁, and Phase₂ may be read and extracted from the plotted impedance phase diagram in step 306.

In an exemplary implementation, analyzing the IPS at the end of each time step may further include determining a cancerous status of an exemplary portion of cancerous tumor 208 based on the calculated IPS. In an exemplary implementation, determining the cancerous status of an exemplary portion of cancerous tumor 208 may include detecting a healthy or necrotic (completely destroyed) region at an exemplary portion of cancerous tumor 208 if the calculated IPS is more than zero or positive and detecting that there is a remaining cancerous region at an exemplary portion of cancerous tumor 208 if the calculated IPS is less than −1. In an exemplary implementation, determining the cancerous status of an exemplary portion of cancerous tumor 208 may include detecting a suspicious region at an exemplary portion of cancerous tumor 208 if the calculated IPS is between about zero and −1.

In an exemplary implementation, destroying of exemplary cancer cells of an exemplary portion of cancerous tumor 208 within each time step (step 308), plotting the impedance phase diagram at end of each time step (step 306), and analyzing the IPS at end of each time step (step 310) may be done in a cycle iteratively after each other until obtaining a positive calculated IPS. In an exemplary embodiment, a positive calculated IPS may define (comprise) a complete destruction of an exemplary portion of cancerous tumor 208; therefore, conducting the cycle may be stopped and the cycle may be repeated for another exemplary portion of cancerous tumor 208. In an exemplary implementation, a cycle of steps 306 to 310 of exemplary method 300 may be carried out portion by portion within cancerous tumor 208 for obtaining complete destroying of exemplary cancerous tumor 208 in a case if exemplary cancerous tumor 208 is a large cancerous tumor. In an exemplary embodiment, each exemplary portion may refer to a confined area of cancerous tissue with a diameter of about 20 mm or less. In an exemplary embodiment, a small cancerous tumor may include one exemplary portion while a large tumor may be more extended within a tissue with higher diameters more than about 20 mm. In another exemplary implementation, a cycle of steps 306 to 310 of exemplary method 300 may be carried out once within cancerous tumor 208 for obtaining complete destruction of exemplary cancerous tumor 208 in a case if exemplary cancerous tumor 208 is a small cancerous tumor with a diameter of about 20 mm or less. In an exemplary implementation, for a large cancerous tumor with a diameter of more than about 20 mm, a cycle of steps 306 to 310 of exemplary method 300 may be carried out in a first exemplary portion with a diameter of about 20 mm or less, and after reaching a positive IPS for the first exemplary portion, a next cycle of steps 306 to 310 of exemplary method 300 may be carried out in a second exemplary portion. This process may be continued until complete destruction of the large cancerous tumor.

In an exemplary implementation, steps 306 to 310 of exemplary method 300 may be carried out in less than one hour, including a complete destruction of exemplary cancerous tumor 208 and real-time monitoring of progress of tumor destruction by electrical impedance phase monitoring within exemplary cancerous tumor 208.

In an exemplary implementation, steps 306 to 310 of exemplary method 300 may be carried out by processing unit 206 utilizing electrical probe 100, impedance analyzer device 202, and DC voltage generator 204. In an exemplary implementation, processing unit 206 may include a memory having processor-readable instructions stored therein and a processor. The processor may be configured to access the memory and execute the processor-readable instructions.

In an exemplary implementation, the processor may be configured to perform a method by executing the processor-readable instructions. In an exemplary implementation, the method may include conducting steps 306 to 310 of exemplary method 300. In an exemplary implementation, the method may include measuring the set of electrical impedance phase values from an exemplary portion of cancerous tumor 208 at a respective set of pre-determined time steps by applying an alternating current (AC) voltage in a sweeping range of frequencies to the first electrode 102 and the second electrode 104 at end of each time step utilizing impedance analyzer device 202 and plotting the impedance phase diagram including the measured set of electrical impedance phase values versus the sweeping range of frequencies (step 306), destroying of exemplary cancer cells of an exemplary portion of cancerous tumor 208 by electrolyzing peripheral medium surrounding cancer cells of the cancerous tumor within each time step by applying a direct current (DC) voltage between the first electrode 102 and the second electrode 104 utilizing DC voltage generator 204 (step 308), and calculating the IPS at end of each time step and stopping destroying of exemplary cancer cells if the complete destruction of an exemplary portion of cancerous tumor 208 is obtained, including obtaining a positive IPS at end of a time step (step 308).

In an exemplary implementation, the processor may be further configured to record and communicate the measured set of electrical impedance phase values from an exemplary portion of cancerous tumor 208 and the calculated IPS at end of each time step to an individual or an expert who may utilize processing unit 206. In an exemplary implementation, the processor may be further configured to stop destroying of exemplary cancer cells by stopping electrolyzing peripheral medium surrounding the cancer cells of an exemplary portion of cancerous tumor 208 if the complete destruction of an exemplary portion of cancerous tumor 208 is achieved. In an exemplary implementation, stopping electrolyzing peripheral medium surrounding the cancer cells of an exemplary portion of cancerous tumor 208 may include ceasing to apply the DC voltage between the first electrode 102 and the second electrode 104. In an exemplary implementation, the complete destruction of an exemplary portion of cancerous tumor 208 may include comparing the calculated IPS with zero at end of each time step and detecting the complete destruction of an exemplary portion of cancerous tumor 208 if the calculated IPS is more than zero (positive). In an exemplary implementation, the processor may be further configured to send an alarm of complete destruction of an exemplary portion of cancerous tumor 208 if a calculated IPS at one time step is positive.

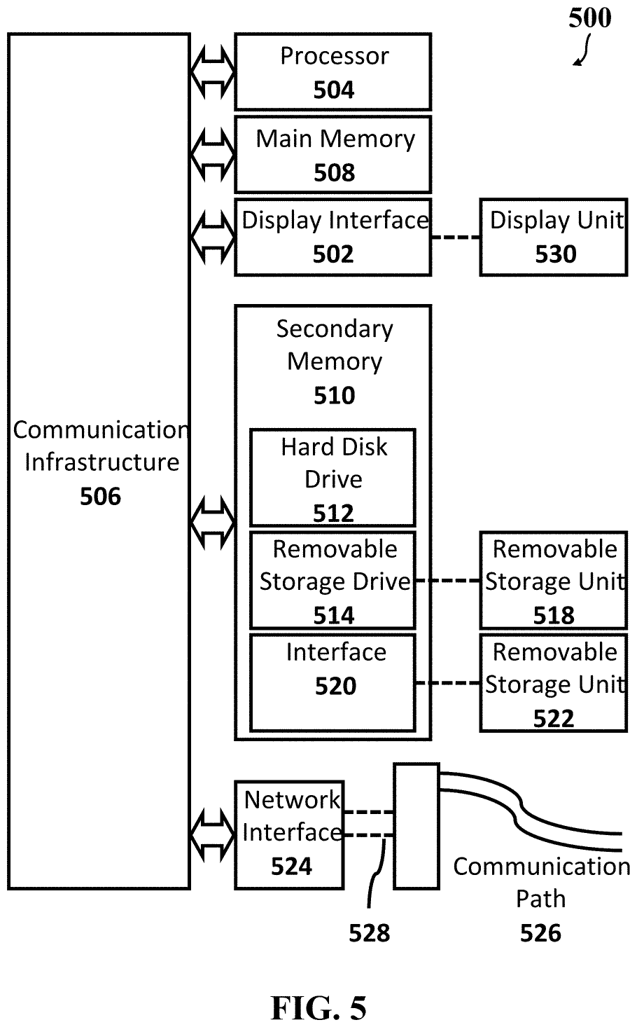

FIG. 5 shows an example computer system 500 in which an embodiment of the present disclosure, or portions thereof, may be implemented as computer-readable code, consistent with one or more exemplary embodiments of the present disclosure. For example, computer system 500 may include an example of processing unit 206, and steps 306, 308, and 310 of exemplary flowchart 300 presented in FIG. 3A, may be implemented in computer system 500 using hardware, software, firmware, tangible computer readable media having instructions stored thereon, or a combination thereof and may be implemented in one or more computer systems or other processing systems. Hardware, software, or any combination of such may embody any of the modules and components in FIG. 2 and FIG. 3A.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the present disclosure is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 504 may be a special purpose or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 504 may be connected to a communication infrastructure 506, for example, a bus, message queue, network, or multi-core message-passing scheme.

In an exemplary embodiment, computer system 500 may include a display interface 502, for example a video connector, to transfer data to a display unit 530, for example, a monitor. Computer system 500 may also include a main memory 508, for example, random access memory (RAM), and may also include a secondary memory 510. Secondary memory 510 may include, for example, a hard disk drive 512, and a removable storage drive 514. Removable storage drive 514 may include a floppy disk drive, a magnetic tape drive, an optical disk drive, a flash memory, or the like. Removable storage drive 514 may read from and/or write to a removable storage unit 518 in a well-known manner. Removable storage unit 518 may include a floppy disk, a magnetic tape, an optical disk, etc., which may be read by and written to by removable storage drive 514. As will be appreciated by persons skilled in the relevant art, removable storage unit 518 may include a computer usable storage medium having stored therein computer software and/or data.

In alternative implementations, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

Computer programs (also called computer control logic) are stored in main memory 508 and/or secondary memory 510. Computer programs may also be received via communications interface 524. Such computer programs, when executed, enable computer system 500 to implement different embodiments of the present disclosure as discussed herein. In particular, the computer programs, when executed, enable processor device 504 to implement the processes of the present disclosure, such as the operations in method 300 illustrated by FIG. 3A, discussed above. Accordingly, such computer programs represent controllers of computer system 500. Where an exemplary embodiment of method 300 is implemented using software, the software may be stored in a computer program product and loaded into computer system 500 using removable storage drive 514, interface 520, and hard disk drive 512, or communications interface 524.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

### Example 1: Fabrication of a Single-Needle Electrical Probe with Two Integrated Needle Electrodes

In this example, an exemplary probe similar to probe 100 was designed and fabricated. Needle of a peripheral venous catheter with gauge 14 was used as an exemplary cathode electrode similar to electrode 102, and a small plastic cannula covered the needle in role of an insulator similar to insulator 106 to prevent electrolysis on non-targeted internal organs and skin. A platinum-iridium (9:1) needle with a thickness of about 250 μm was utilized as an exemplary anode electrode similar to electrode 104, and was inserted inside the cathode electrode. Initially, surface of the platinum-iridium needle was roughened in nanometer scale. Then, a desired part of the platinum-iridium needle except a respective distal end was coated with an electrically insulating layer similar to second electrically insulating layer 108. Afterwards, the platinum-iridium needle was placed inside the cathode electrode for utilizing as an anode electrode in EChT applications.

For nanoroughening surface of the platinum-iridium needle, both wet etching (utilizing an Aqua Regia solution) and dry etching (utilizing fluoride-based gas) were applied for platinum etching, and both chemically and physically enhanced effects of etching were examined. First, the needle in Aqua Regia (8:1 mixture of HCl and HNO₃), which is a wet etchant for Pt, was kept on a hotplate at around 60° C. throughout an etching process for about 180 seconds. Shortly after initiating, Aqua Regia begins to effervesce and develops a dark orange color. After removal from the etchant the sample was washed with deionized water and dried with compressed air. For dry etching, after investigation of different ranges for various parameters, optimized parameters were used to achieve a uniform porous platinum needle with Reactive Ion Etching (RIE), including: RF power=250 W, processing mixture=SF₆ (150 sccm)+O₂ (150 sccm), processing time=20 minutes, and processing pressure=20 mTorr.

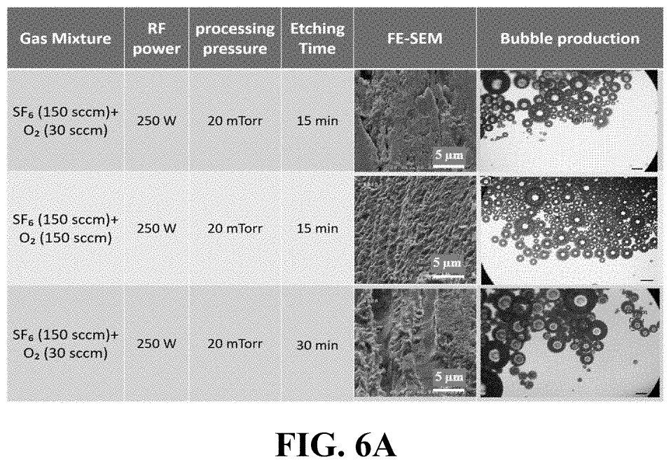

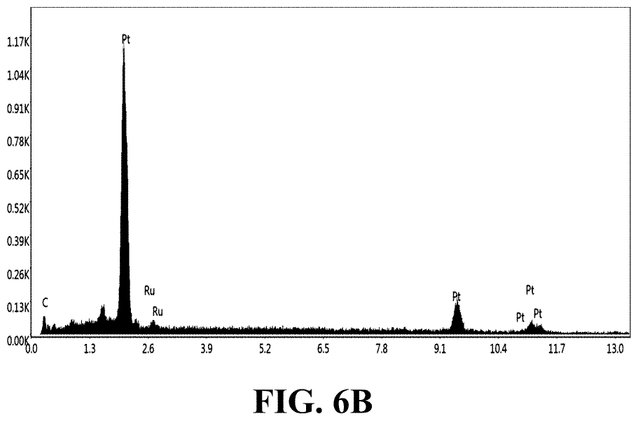

Various parameters for making porosity on a Pt needle surface utilizing an electrochemical approach were tested. Various parameters for electrochemical porous of Pt needles included gas mixture, power of radiofrequency generator of reactive ion etching, pressure of porous process, and time of process. FIG. 6A shows various parameters for electrochemically production of porous texture on Pt needles, field emission-scanning electron microscopy (FE-SEM) and bubble production results for different fabricated nanoporous Pt needles at different conditions, consistent with one or more exemplary embodiments of the present disclosure. As shown in FIG. 6A, density of produced microbubbles generated by porous Pt electrodes with parameters of radio frequency (RF) power=250 W, processing mixture=SF₆ (150 sccm) and O₂ (150 sccm), processing time=20 minutes, and processing pressure=20 mTorr, showed the most uniform porous platinum needle by which bubble generation was started in lower voltages (<1.5 V). Also, microbubbles generated in similar stimulated voltage (4 V) showed that a mean size of bubbles generated by porous Pt electrodes with optimized parameters was less than about 40 μm while for other fabricated electrodes (bare Pt electrode and other porous Pt electrodes (mentioned in FIG. 6A)) was more than about 100 μm.

Additionally, energy dispersive X-ray spectroscopy (EDS) was carried out for porous needles to show that making nanoporous texture on needle surface wouldn't make alloy in Pt material. FIG. 6B shows EDS spectra of an exemplary porous Pt needle fabricated with optimized parameters, including: RF power=250 W, processing mixture=SF₆ (150 sccm)+O₂ (150 sccm), processing time=15 minutes, and processing pressure=20 mTorr, consistent with one or more exemplary embodiments of the present disclosure. As expected, there are just Pt and Ru peaks in the EDS images of porous Pt needle similar to bare Pt needle.

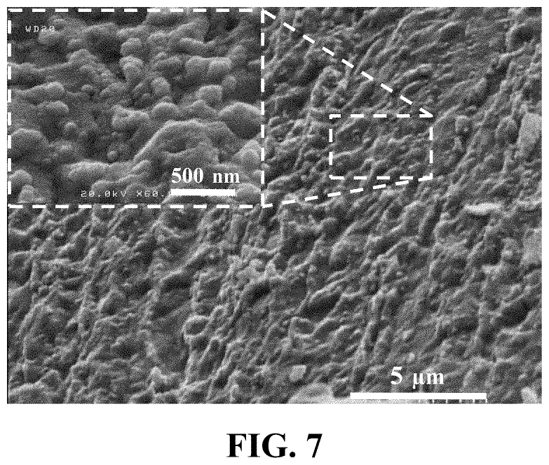

Surface profiles of the etched Pt needle were investigated using FE-SEM. FIG. 7 shows FE-SEM images of an exemplary nanoporous Pt needle, consistent with one or more exemplary embodiments of the present disclosure. It may be observed that uniform nanopores with an average size of less than about 300 nm for each nanopores may be formed on surface of the Pt needle. It should be noted that uniformly formed small nanopores may lead to forming small bubbles while electrolyzing a portion of a cancerous tumor utilizing the fabricated probe with the nanoporous Pt needle. The small bubbles may lead to more effective destroying of cancer cells within the portion of the cancerous tumor.

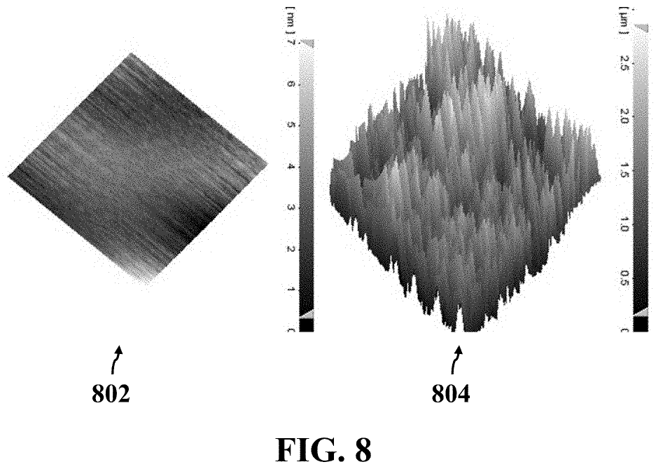

Surface roughness of Pt needles after etching was measured by atomic force microscopy (AFM). FIG. 8 shows AFM topographies of an exemplary bare Pt needle (image 802) and an exemplary nanoporous Pt needle (image 804), consistent with one or more exemplary embodiments of the present disclosure. RMS (root mean square) roughness of nanoporous Pt electrode was more than about two orders of magnitude of a non-porous (bare) Pt electrode. Surface morphology (electrode surface roughness) may be an important issue for interactive electrode performance because it may reduce treating time and electrical power for an efficient electrolysis.

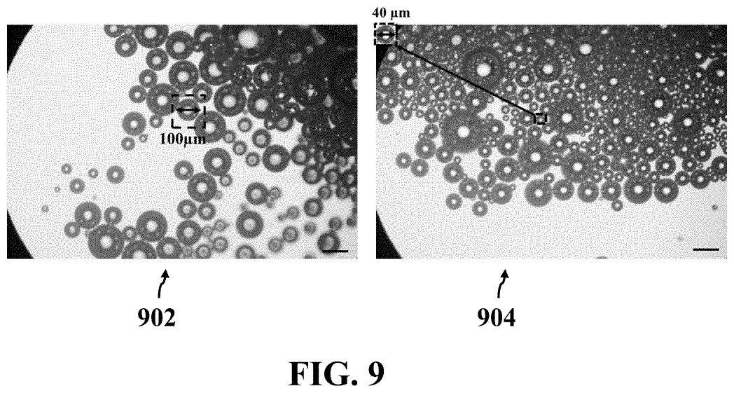

FIG. 9 shows generation of microbubbles in saline by an exemplary bare Pt needle (image 902) and a nanoporous Pt needle (image 904) at about 4 volts, consistent with one or more exemplary embodiments of the present disclosure. It may be observed that density of produced microbubbles generated by an exemplary porous Pt electrode was higher than a bare Pt electrode on a saline solution. Furthermore, bubble generation was started in lower voltages (about 1.5 V) for an exemplary porous Pt electrode in comparison with a bare Pt electrode. Also, statistical portray on size of generated microbubbles in the same stimulated voltage (about 4 V) showed that mean size of bubbles generated by a porous Pt electrode was less than about 40 μm while it was more than about 100 μm for bare Pt electrodes (FIG. 9). Using porous Pt electrodes resulted in a meaningful increase in number and decrease in size of generated microbubbles even by applying lower electrical power.

### Example 2: In-Vivo Evaluations of Exemplary Fabricated Electrical Probe

In this example, efficiency of an exemplary probe similar to probe 100 for EChT of cancerous tumors and its possible side effects on normal tissues during EChT was investigated. Two cohorts of female BALB/C mice and one cohort of female rats at ages of about 6-8 weeks were selected (10 mice in each cohort) and tumorized by 4T1 (Mouse type) breast cancer cells.

For cell culturing, 4T1 mouse breast cancer cell lines were obtained from a National cell bank (NCBI). The obtained 4T1 cell lines were maintained in an incubator at about 37° C. (about 5% CO₂, about 95% air) in a Roswell Park Memorial Institute (RPMI) medium, supplemented with about 5% fetal bovine serum and about 1% penicillin/streptomycin. The fresh medium was replaced every day. All cell lines were tested and found negative for Mycoplasma contamination. The cells were detached from the plates by trypsin and counted by a Nicobar laam (Nicobar laam is Haemocytometer Neubauer that is a chamber in a slide for cell counting in laboratory).

Then, cancerous tumors were formed in animal models. For mice models, female inbred BALB/c mice at age of about 6-8 weeks were purchased. They were kept at about 22-24° C. with about 12 hours light/dark cycle in an utterly designed pathogen-free isolation facility and allowed them to adapt for one week before tests. About 3×10⁶ 4 T1 cells/200 μl in a logarithmic growth phase were subcutaneously (s.c.) injected into backside of BALB/c mice. Tumor size was measured using a portable sonogram. When a tumor mass formed and reached a desired size for each group, the mice were divided into the desired groups, and further treatment was conducted. Every 5 days, tumor size was measured by ultrasonography technique until the end day. A tumor volume was calculated according to equation (1) below:

$$Tumor volume = ( \frac{4}{3} ) * Π * ( \frac{Length}{2} ) * ( \frac{Length}{2} ) * ( \frac{Depth}{2} ) \qquad ( 1 )$$

Additionally, rats models were healthy young adult female (nulliparous and non-pregnant) Fischer 344 rats. Rats were acclimated for seven days before tumor cell injection and were housed individually in separate cages when not receiving experimental treatments. All Groups were anesthetized by injection with a combination of ketamine (75 mg/kg) and acepromazine (2.5 mg/kg) before electrode implantation. Tumors were induced in all animals through subcutaneous (s.c.) injection of sacrificed tumor mass of mice in the abdominal area of rats. Tumor growth was monitored daily and tumor volume was calculated according to equation (1).

The first and second groups consisted of mice with tumor sizes of about 100-300 mm³ and 300-500 mm³, and the third one was rats with tumor sizes of more than about 700 mm³. Half of the animals from each group were treated with an exemplary probe with a nanoporous Pt anode electrode and another half were treated with an exemplary probe with a non-porous Pt anode electrode. Exemplary probe was inserted into tumors from skin under with guide of ultrasonography imaging. An outer needle entered the tumor mass horizontally; then, an inner needle was pushed into an end site of the tumor under the vision of sonography. Then, destroying of cancer cells by electrolysis was started which may livelily be monitored either by monitoring electrical impedance response utilizing the same probe or by ultrasonography.

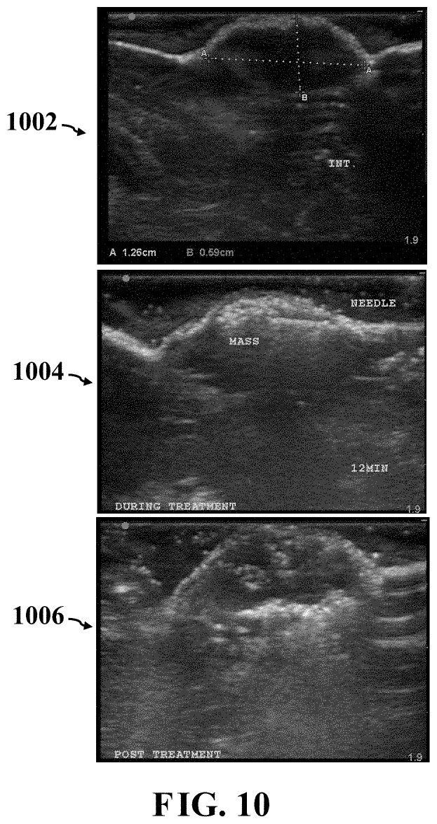

FIG. 10 shows ultrasonography images of a treated mouse utilizing an exemplary probe with nanoporous Pt anode electrode, including tumor mass before (image 1002), tumor cite during (image 1004), and tumor cite after (image 1006) treatment, consistent with one or more exemplary embodiments of the present disclosure. Disruption of the tumor mass structure due to the treatment is visible in FIG. 10. Presence of some gas bubbles under the vision of sonography (due to formation and release of Cl and H based gases) was the first indication of treatment initiation to monitor the electrolysis process, distribution and abundances of bubbles became so intense after about 10 min. Hence, it may be hard to monitor events in the mass in later times. Intra treating impedance spectroscopy, as proposed and disclosed in exemplary method 300, may be an appropriate alternative to check a biological sate of a treated tumor by calibrated impedance signals.

Moreover, ultrasonography images of other organs of the treated mouse such as kidney, heart, and liver during the process of treatment and after that were checked (not illustrated). It was observed that there was no damage to the mouse's organs such as the kidney, heart (the heartbeat of the mice was normal), and liver during the process of treatment and after that, and the mice were completely healthy. Only the tumor mass has been affected by the treatment. Also, histological patterns of mouse's body organs, such as colon, kidney, liver, lung, and spleen were checked for possible side effects.

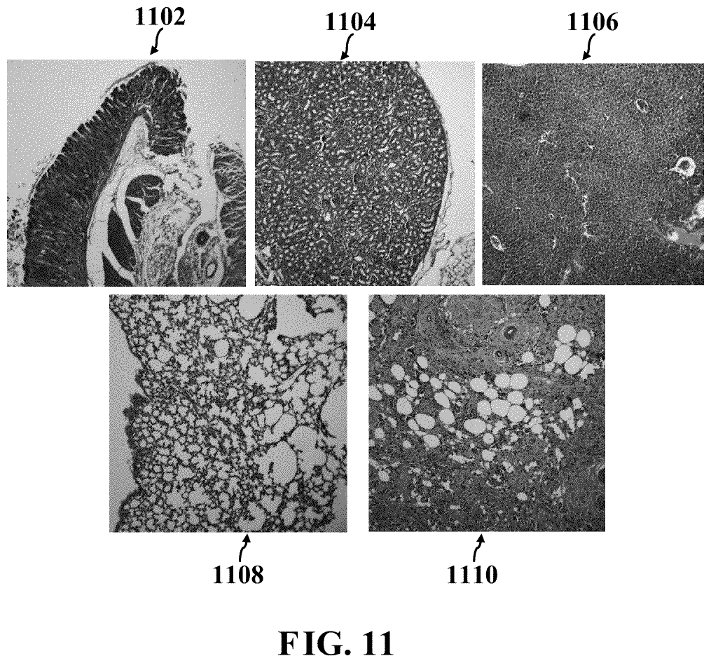

FIG. 11 shows histological patterns of treated mouse's body organs, including colon (image 1102), kidney (image 1104), liver (image 1106), lung (image 1108), and spleen (image 1110), consistent with one or more exemplary embodiments of the present disclosure. No damage to the mouse's body organs was observed utilizing exemplary probe with the nanoporous Pt anode electrode. It was observed that utilizing exemplary fabricated probe with a nanoporous Pt electrode biased to low voltages resulted in selective destructive effects on tumor region with neither skin damage nor pathological effects on main organs such as liver, peritoneal cavity, etc. Hence, a localized selective acute tumor therapy in deep regions of body may be achieved without inducing any side effects to upper or deeper tissues and organs.

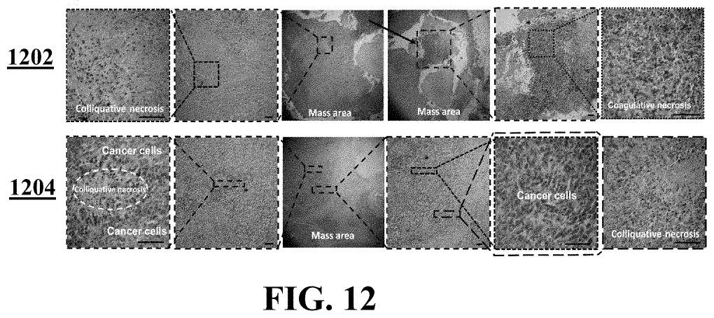

To present comparative effect of non-porous/porous Pt electrodes in efficiency and side effects of EChT, data of two mice from cohort 2 (second treated groups consisted of mice with tumor sizes of 300˜500 mm3) is shown in FIG. 12. FIG. 12 shows histological patterns of Hematoxylin and eosin (H&E) assay for treated mass area utilizing an exemplary non-porous Pt anode electrode (image 1202) and an exemplary porous Pt anode electrode (image 1204), consistent with one or more exemplary embodiments of the present disclosure. A current level of about 4 mA was made in the tumor side just by applying about 1.5 V to the porous Pt electrode while about 6.5 V was required to achieve the same current by the non-porous Pt electrode. Since electrical current plays a crucial role in efficiency of EChT, so with about 80% lower electrical power, a therapeutic efficiency similar to a situation of using a non-porous Pt electrode was achieved by porous Pt electrodes. Depletion of lower electrical power in body of a live person or animal would significantly reduce many probable risk factors such as cardiac perturbation or skin burning. One day after the therapy, the tumors and other organs of both two mice were dissected for histological analysis. H&E staining results showed distinct areas of necrosis around the two electrodes.

As can be seen in the FIG. 12, the porous needle showed a more extended necrotic pattern in the whole area treated with exemplary probe. Also, there were some active cancer cells in the treated areas of non-porous Pt needle. Porous Pt needle with 4.3 times lower electrical power than the non-porous Pt needle showed much greater therapeutic effects. Therefore, higher treatment efficiency may be achieved with lower treating voltages utilizing a nanoporous electrode. It should be noted that EChT method makes a region surrounding an anode electrode very acidic (a pH of about 2) and a region surrounding a cathode electrode strongly alkaline (a pH of about 12). At these extreme, pH changes tissue proteins, it becomes denaturized, the cell structure collapses, and the cell finally dies. This change in pH, in combination with released chlorine gas, and hydroxyl free radicals' may result in a locally cytotoxic microenvironment that may induce coagulative and colliquative necrosis in an area around the anode and cathode electrodes. As a result of a loss of biochemical function, cell nuclei were not stained at the region around the cathode. No cell membranes were detected. As a result of cell lysis, the histology image revealed the homogenization of (light) cytoplasm and the loss of the normal lobular structure, in contrast, the cytoplasm in the necrotic area around the anode was found to be strongly eosinophilic. The morphological structure of the cell nuclei was still intact, and the cell membranes were visible.

Experimentally achieved optimal current and voltage parameters for treatment of mice and rats utilizing exemplary probe with non-porous/porous Pt anode electrode are presented in Table 1. Up to 5 days after treatment, inflammation and swelling tumor residues by the immune system may be occurred; hence, tumor volume changes were measured after 5 days. If cancerous mass was still observed in sonography images, the second treatment was performed and the animal was monitored again in 5 days. Weight, temperature, visual appearance, and behavior of all animals were monitored daily. The control animal did not exhibit any weight loss during the tests, while animals in treated groups lost up to about 10% of their weight. However, they recovered within about 10-15 days.

TABLE 1
Optimized parameters for the treatment of animal models utilizing
exemplary electrical probe with non-porous/porous Pt anode electrode

| | V (v) | | I | Time |
|---|---|---|---|---|
| Animal type | Non-porous Pt | Porous Pt | (mA) | (min) |
| Mice | 6.5-9 | 1.5-2.5 | 4-6 | 15-20 |
| Rat | 11-14 | 2.8-4 | 7-9 | 25-30 |

Table 2 represents detailed information of EChT parameters utilizing exemplary fabricated electrical probe for three groups of mice. Tumors were disappeared in all mice (5 mice) of the first group treated with porous Pt anode electrode before the second treatment (up to 5 days after the first treatment) without any recurrence. But, 2 of 5 (40%) mice that treated with non-porous Pt needle had a recurrence (ID 7) or tumor remaining (ID 9) (with a tumor size of 15.5 mm³ and 56.8 mm³, respectively) and required to second treatment. In the second group, 3 of 5 (60%) mice had been treated by non-porous Pt anode electrode required for the second treatment. All of the mice had been treated by a porous Pt electrode became free of tumor. In the third group which had larger tumor volumes, 3 of 5 (60%) and 1 of 5 (20%) rats had been treated by non-porous and porous Pt needle required to second and even third EChT, respectively. Similar responses were also observed in rat samples.

TABLE 2
Detailed information of EChT parameters utilizing exemplary fabricated electrical
probe for three groups with different tumor sizes

| | | | Primary | | | | | |
|---|---|---|---|---|---|---|---|---|
| | | Type of | tumour | First treatment parameters | | | | State of |
| | | Pt anode | volume | V | I | Time | Number of | animal up to |
| Group | ID # | electrode | (mm³) | (v) | (mA) | (min) | treatments | 180 days |
| 1 | 1 | NP | 112.5 | 6-6.5 | 4 | 15 | 1 | A |
| 1 | 2 | P | 114.8 | 1.6-2 | 4 | 15 | 1 | A |
| 1 | 3 | NP | 129.8 | 6-6.5 | 4 | 15 | 1 | A |
| 1 | 4 | P | 141.6 | 1.6-2 | 4 | 15 | 1 | A |
| 1 | 5 | NP | 160.3 | 6-6.5 | 4 | 15 | 1 | A |
| 1 | 6 | P | 180.6 | 1.6-2 | 4 | 15 | 1 | A |
| 1 | 7 | NP | 199.3 | 7-7.5 | 5 | 15 | 2 (R) | A |
| 1 | 8 | P | 228 | 2-2.2 | 5 | 15 | 1 | A |
| 1 | 9 | NP | 251.2 | 7-7.5 | 5 | 15 | 2 (TR) | D (Day 6) |
| 1 | 10 | P | 285.7 | 2-2.2 | 5 | 15 | 1 | A |
| 2 | 11 | NP | 325.6 | 7.5-8.5 | 5.5 | 20 | 1 | A |
| 2 | 12 | P | 329.8 | 2.1-2.3 | 5.5 | 20 | 1 | A |
| 2 | 13 | NP | 347.8 | 7.5-8.5 | 5.5 | 20 | 1 | A |
| 2 | 14 | P | 349.6 | 2.1-2.3 | 5.5 | 20 | 1 | A |
| 2 | 15 | NP | 359.5 | 7.5-8.5 | 5.5 | 20 | 2 (R) | A |
| 2 | 16 | P | 383 | 2.1-2.3 | 5.5 | 20 | 1 | A |
| 2 | 17 | NP | 424.3 | 8-9 | 6 | 20 | 2 (R) | D (Day 11) |
| 2 | 18 | P | 441.6 | 2.3-2.5 | 6 | 20 | 1 | A |
| 2 | 19 | NP | 474.9 | 8-9 | 6 | 20 | 2 (TR) | D (Day 7) |
| 2 | 20 | P | 490.2 | 2.3-2.5 | 6 | 20 | 1 | A |
| 3 | 21 | NP | 710 | 11-12 | 7 | 25 | 1 | A |
| 3 | 22 | P | 798.5 | 2.8-3 | 7 | 25 | 1 | A |
| 3 | 23 | NP | 828.07 | 11.5-13 | 8 | 30 | 1 | A |
| 3 | 24 | P | 897.6 | 3-3.5 | 8 | 30 | 1 | A |
| 3 | 25 | NP | 925.3 | 11.5-13 | 8 | 30 | 3 (TR) | D (Day 16) |
| 3 | 26 | P | 1015.4 | 3-3.5 | 8 | 30 | 1 | A |
| 3 | 27 | NP | 1431.8 | 13-14 | 9 | 30 | 2 (R) | A |
| 3 | 28 | P | 1668.5 | 3.5-4 | 9 | 30 | 1 | A |
| 3 | 29 | NP | 1885.6 | 13-14 | 9 | 30 | 3 (TR) | D (Day 15) |
| 3 | 30 | P | 2252.9 | 3.5-4 | 9 | 30 | 2 (TR) | A |

(Note:
D = Death, A = Alive, R = Recurrence; TR = Tumor remaining up to 5 days after each treatment)

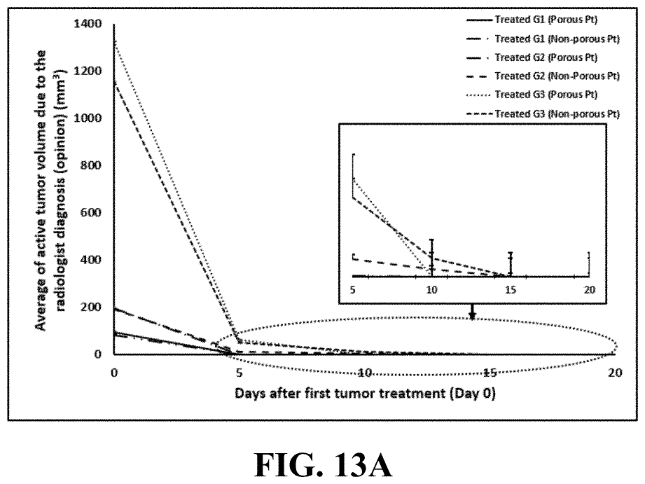

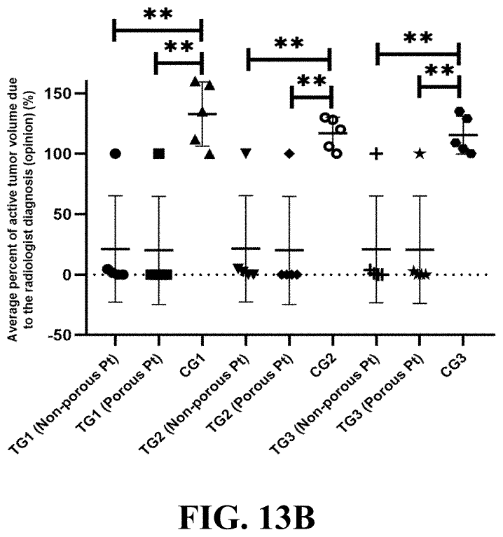

Regarding Tables 1 and 2, a high therapeutic efficiency with fewer steps of therapy and lower transmitted electrical power, and higher tumor destruction was obtained utilizing an exemplary porous Pt anode electrode, with the least tumor recurrences. The depleted electrical power for exemplary probe with porous Pt anode electrode was less than about 36 mW in about 30 min in comparison with an electrical power of about 126 mW for exemplary non-porous Pt anode electrode. So, the porous Pt anode electrode had better efficacy in tumor destruction without any tumor recurrence or residue as may be observed in FIGS. 13A and 13B. FIG. 13A shows average of active tumor volume due to the radiologist diagnosis (opinion) of treated groups from the first treatment monitored by ultrasonography imaging, consistent with one or more exemplary embodiments of the present disclosure. FIG. 13B shows differences in mean tumor size between treatment with non-porous/porous Pt anode electrode and control groups (TG1 (Non-porous Pt) and TG1 (Porous Pt) versus CG1: p<0.0013 and p<0.0011, TG2 (Non-porous Pt) and TG2 (Porous Pt) versus CG2: p<0.0087 and p<0.0074, and TG3 (Non-porous Pt) and TG3 (Porous Pt) versus CG3: p<0.0096 and p<0.0092) up to twenty days after the first treatment for each group, consistent with one or more exemplary embodiments of the present disclosure.

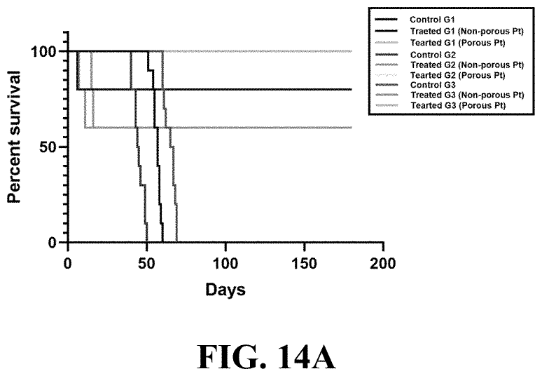

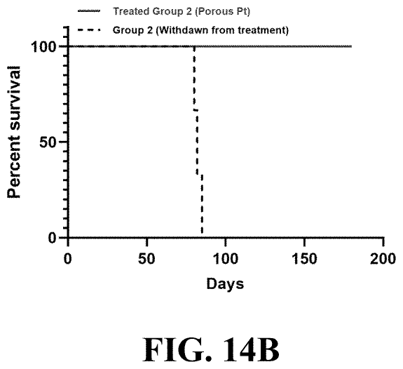

FIGS. 14A and 14B depict Kaplan-Meier survival curves in 3 groups of animals in this example. FIG. 14A shows Kaplan-Meier survival curves of 3 groups of treatments (n=10 mice per group), consistent with one or more exemplary embodiments of the present disclosure. The maximum survival rate belongs to treated mice utilizing exemplary probe with porous Pt anode electrode. FIG. 14B shows Kaplan-Meier survival curves of group 2 (G2) treated mice with porous Pt anode electrode and withdrawn from treatment (n=3 mice per group), consistent with one or more exemplary embodiments of the present disclosure. It may be observed that withdrawal of mice (for example mice from G2) causes the recurrence of tumors and mortality.

### Example 3: EChT Assisted with Intra-Therapeutically Electrical Impedance Monitoring

In this example, an exemplary system similar to system 200 was utilized for conducting a cancer treatment process similar to method 300 for treating cancerous tumors in both rats and human. Impedance monitoring was used as a method for screening EChT treatment and impedance values of an exposed tissue to EChT was measured by exemplary electrical probe 100, before, during, and after the EChT treatment. When a measured impedance of a treated zone reached to an impedance of a necrotic tissue (that had been derived by calibrating impedance values of vital and necrotic mice/human tumors), a length of anode electrode was readjusted and entered to a non-treated zone, and again measuring/treating cycle was started. Based on this exemplary method, all depth of the tumor may be treated in an efficient time. As the presence of gas, generated during the EChT, may perturb contrast of ultrasonography imaging to monitor therapeutic efficiency, impedance-based monitoring may be a more efficient alternative. Monitoring of impedance values and EChT treatment was done utilizing the same electrical probe similar to exemplary electrical probe 100.

Table 3 shows tumor mass impedance results utilizing exemplary electrical probes with porous and non-porous Pt electrodes before, during, and after EChT treatment for two rats of rats which were tumorized according to EXAMPLE 2 described herein above. Applied voltage and current for EChT treatment on rat, were 11-14 V and 2.8-4 V with constant current 7-9 mA for non-porous and porous Pt electrodes, respectively.

TABLE 3
Tumor mass impedance results utilizing exemplary electrical
probes with porous and non-porous Pt electrodes before,
during, and after EChT treatment

| Time of | | | | |
|---|---|---|---|---|
| recording | Z (1 kHz) | | IPS (Impedance | |
| (minutes after | (ohm) | | phase slope between | |
| starting of | Non-porous | Porous | 100 kHz and 500 kHz) (degree) | |
| EChT) | Pt | Pt | Non-porous Pt | Porous Pt |
| 0 (Before | 1774 | 749 | −2.86 | −8.2 |
| treatment) | | | | |
| 2 | 1680 | 658 | −1.32 | −3.8 |
| 5 | 1406 | 476 | −0.87 | −2.24 |
| 10 | 1049 | 304 | 0.51 | 1.49 |
| 15 | 642 | 207 | 2.48 | 6.77 |
| 20 | 315 | 108 | 3.81 | 10.43 |
| 25 (After | 221 | 86 | 4.66 | 13.90 |
| treatment) | | | | |

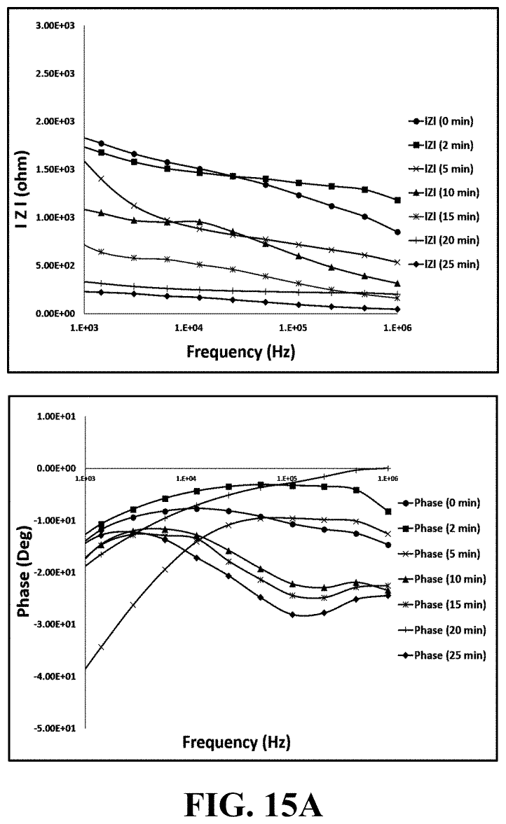

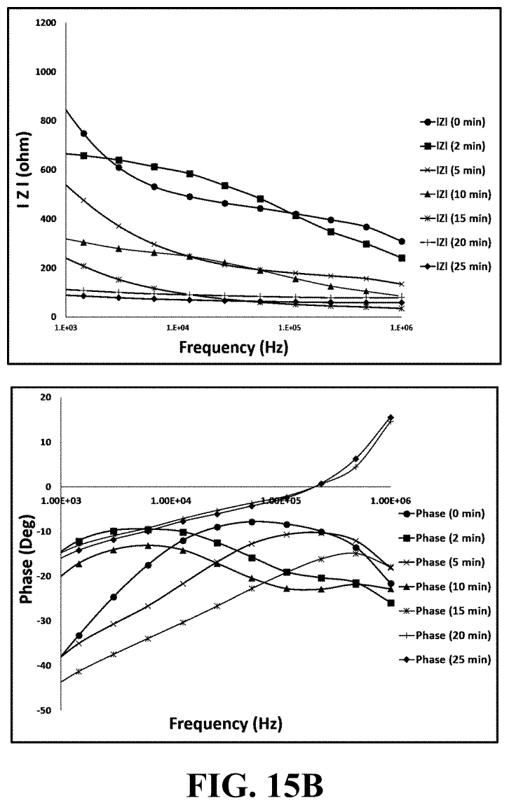

FIG. 15A shows impedance magnitude and phase diagram for a rat treated by EChT with a bare Pt anode electrode, consistent with one or more exemplary embodiments of the present disclosure. FIG. 15B shows impedance magnitude and phase diagram for a rat treated by EChT with a porous Pt anode electrode, consistent with one or more exemplary embodiments of the present disclosure. It may be observed that a difference between impedance amplitude and phase in distinguishing between healthy and cancerous lesions is about 2.36-3.45 and 2.7-3 times utilizing the porous electrode compared to the non-porous electrode, respectively. Hence, monitoring a cancerous tumor destruction and detection of a complete destruction of the cancerous tumor may be more distinguishable utilizing a porous electrode in comparison with a non-porous electrode.

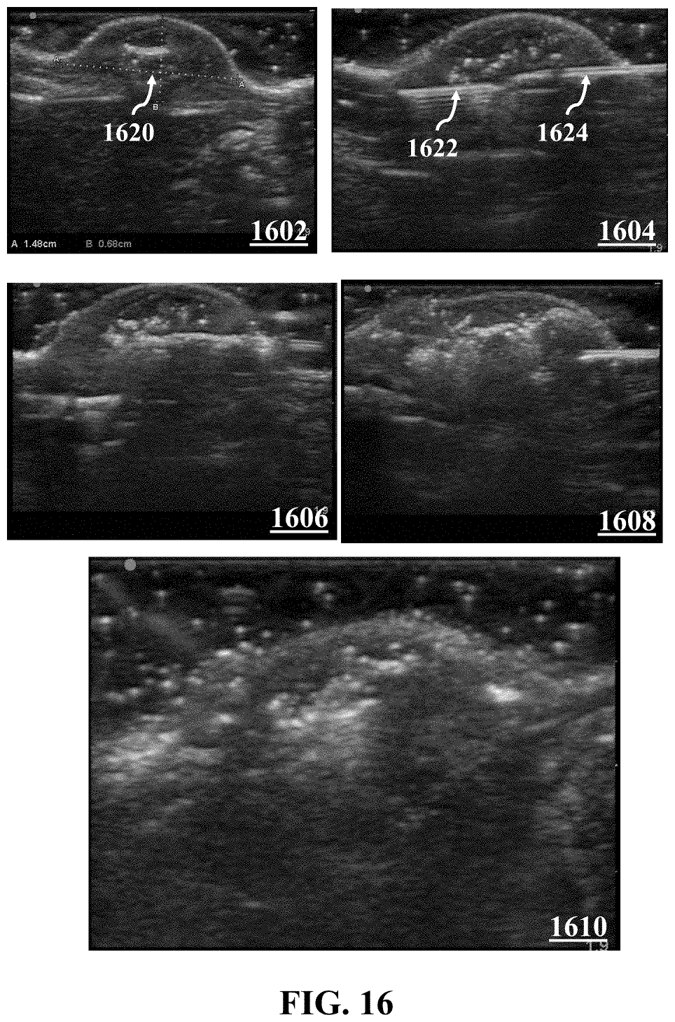

FIG. 16 shows ultrasonography images of a tumor site of a treated rat by EChT utilizing an electrical probe with porous Pt anode electrode, including before treatment (image 1602), the electrical probe placed inside the tumor (image 1604), about 2 minutes after applying DC (image 1606), about 15 minutes after starting treatment (image 1608), and immediately after treatment (25 min) (image 1610), consistent with one or more exemplary embodiments of the present disclosure. It may be observed in image 1602 that tumor 1620 invaded underlying structures of liver. In image 1604, cathode electrode 1624 and porous Pt anode electrode 1622 was inserted into tumor 1620. Immediately after applying DC, changes were visible around the electrical probe and may be clearly seen in image 1606 recorded about two minutes after starting the treatment. But, after 2 minutes, the changes (bubble formation and release of Cl₂ and H₂ gases) become so intense around the electrical probe that it may impossible to monitor events in tumor mass in later times (e.g., 15 minutes after treatment shown in image 1608). Immediately after treatment (about 25 min shown in image 1610), disruption of tumor 1620 structure was visible.

Accordingly, monitoring of electrical impedance during EChT therapy may brightly inform an expert about local destruction of a tumor in each location of the tumor. Hence, after reaching a complete destruction of a portion of the tumor, the expert may move electrodes of an exemplary electrical probe to other areas of the tumor to finally make and monitor the total EChT destruction. Such live impedance recording during cancer therapeutic procedure may help to transfer the least electrical power needed for the destruction of the tumor in every location. While because of tissue damages and bubbles induced during acidification and basification of the tumor in two ends of the electrodes, ultrasonography imaging may show bare patterns from the tumor. In other words, it may be impossible to monitor changes around the electrodes after about 2 minutes in tumor mass region in sonography images.

An example revealed the importance and impact of impedance recording for treatment monitoring. In one case, after EChT treatment disruption of mass structure was visible under sonography images which showed no evidence of tumor cells while impedance monitoring results showed the presence of active tumor inside the mass due impedance to values of mice breast tumor. The EChT treatment was stopped after about 20 minutes for mice case the mass situation was monitored. After about 5 days, traces of tumor mass were seen in ultrasonography images in corroboration with intra-therapeutically impedance recording results.

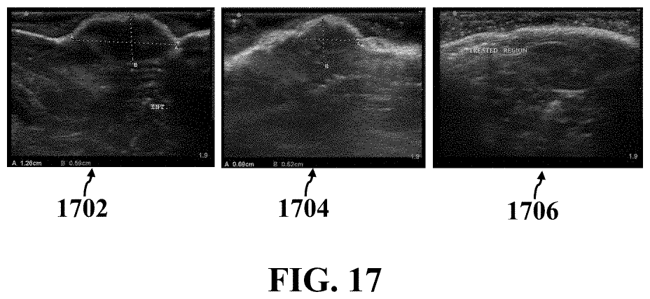

FIG. 17 shows ultrasonography images of tumor cite before treatment (image 1702), EChT treated tumor cite after about five days of first treatment (image 1704), and tumor cite about five days after second treatment with EChT (image 1706), consistent with one or more exemplary embodiments of the present disclosure. It may be observed from image 1704 that after about five days of the first treatment the tumor remained within tissue. Furthermore, about five days after the second treatment no evidence of mass was observed (image 1706).

Additionally, an exemplary system similar to system 200 was utilized for conducting exemplary cancer treatment processes similar to method 300 for treating a patient having Sarcoma and a patient having a malignant breast cancer. Treatments were done utilizing exemplary electrical probes similar to probe 100 with porous Pt electrodes. Table 4 shows tumor's characteristics, treatment parameters, and treatment results for these two patients having Sarcoma and malignant breast tumor, respectively.

TABLE 4
Tumor's characteristics, treatment parameters, and treatment results
for a patient having Sarcoma and a patient having a malignant breast tumor.

| | Patient | |
|---|---|---|
| Parameters | Patient 1 | Patient 2 |
| Patient age | 33 | 49 |
| Tumor type | Spindle cell sarcoma | IDC grade 2 |
| | (Left buttocks) | (10 o'clock in Left |
| | | breast) |
| Primary tumor size (mm²) | 62 * 20 | 25 * 16 |
| Average treating electrical power (mW) | 864 | 876 |
| Total treating time in N sessions (min) | 245 in 3 sessions | 303 in 3 sessions |
| Total efficient transmitted electrical charge | 1069.35 | 1042.32 |
| (A.S) | | |
| Required charge induce 1% necrose in the | 12.96 | 10.42 |
| tumor (A.S) | | |
| Final tumor size after treatment | The primary tumor | 0 (mm²) |
| | divided two masses after | |
| | three sessions: 22.7 * 12.5 | |
| | and 32.8 * 7.4 (mm²) | |
| Percentage of necrosis one month after the | Extensive areas of fibrotic | No evidence of |
| last treatment (Due to permanent | and necrotic changes, | suspicious enhance and |
| pathology/Sonography and MRI report) | about 80-85% of tumoral | tumoral mass (MRI) |
| | tissue (Permanent | |
| | pathology) | |
| Treatment Efficiency (%) | 85 | 100 |
| (EChT induced tumor necrosis (%)) | | |
| Recurrence after three months (Due to | — | No (due to MRI report) |
| permanent pathology/Sonography and | | |
| MRI report) | | |

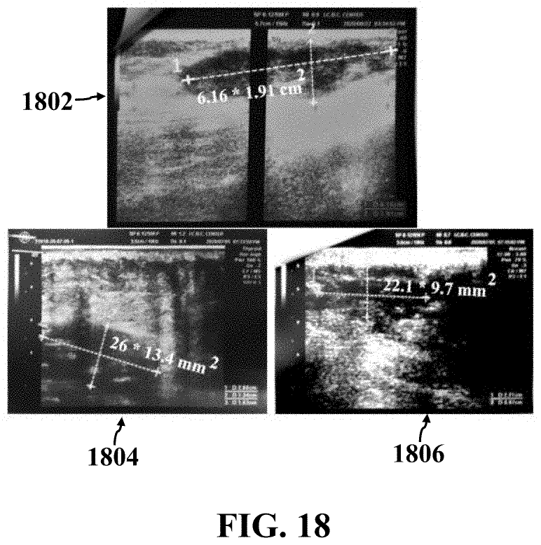

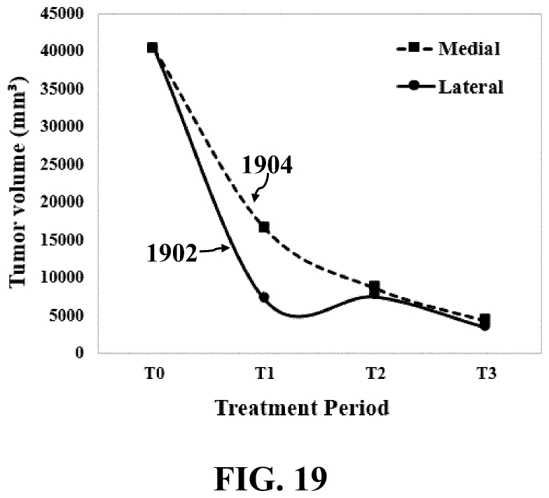

Referring to Table 4, patient 1 had Sarcoma tumor at left buttock. FIG. 18 shows ultrasonography images of Sarcoma tumor of patient 1 before treatment (image 1802) and divided tumor into two parts of lateral part (images 1804) and medial part (images 1806) after three cycles of impedance monitoring-assisted EChT treatment, consistent with one or more exemplary embodiments of the present disclosure. It may be observed that after applying three cycles of EChT treatment of tumor by electrolyzing Sarcoma tumor assisted by monitoring impedance values inside the Sarcoma tumor, the Sarcoma tumor was divided to two parts lateral (images 1804) and medial (image 1806) with an overall reduced size of tumor (26 mmx 13.4 mm for lateral part and 22.1 mmx 9.7 mm for medial part) in comparison with initial size (61.6 mmx 19.1 mm). FIG. 19 shows decreasing trend of tumor volume for lateral part (curve 1902) and medial part (curve 1904), consistent with one or more exemplary embodiments of the present disclosure. An overall reduction of about 60% was observed for tumor size in comparison with initial size.

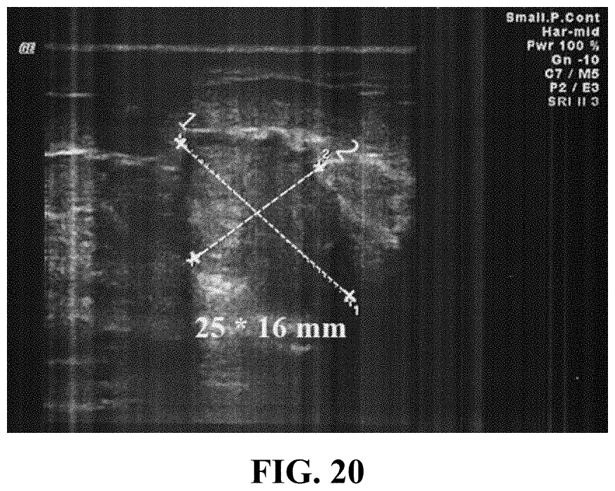

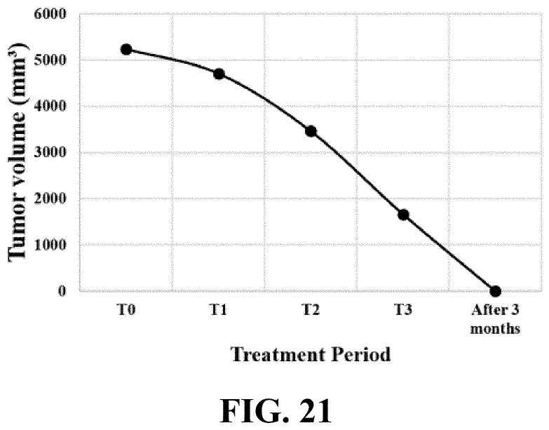

Referring again to Table 4, patient 2 had malignant breast tumor of invasive ductal carcinoma (IDC) grade 2 at 10 o'clock in left breast. FIG. 20 shows ultrasonography images of IDC tumor of patient 2 before treatment, consistent with one or more exemplary embodiments of the present disclosure. It may be observed that tumor had an initial size of 25 mmx 16 mm. After applying three cycles of EChT treatment of tumor by electrolyzing the IDC tumor shown in FIG. 20, where the applied EChT treatment was assisted by monitoring impedance values inside the IDC tumor, the IDC tumor was completely disappeared. FIG. 21 shows decreasing trend of tumor volume for exemplary treated IDC tumor of patient 2, consistent with one or more exemplary embodiments of the present disclosure. It may be seen from FIG. 21 that tumor volume decreased to zero after three cycles of treatments and also no recurrence of tumor was happened after 3 months after treatment.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for destroying a cancerous tumor, comprising:
putting two electrodes of an electrical probe in contact with a portion of the cancerous tumor;
plotting an impedance phase diagram by measuring a set of electrical impedance phase values from the portion of the cancerous tumor at a respective set of pre-determined time steps by applying an alternating current (AC) voltage in a sweeping range of frequencies to the two electrodes at end of each time step of the respective set of pre-determined time steps utilizing an impedance analyzer device;
destroying cancer cells of the portion of the cancerous tumor within each time step of the respective set of pre-determined time steps by electrolyzing peripheral medium surrounding the cancer cells of the portion of the cancerous tumor, the electrolyzing peripheral medium comprising applying a direct current (DC) voltage between the two electrodes utilizing a DC voltage generator; and
stopping destroying of the cancer cells responsive to a complete destruction of the portion of the cancerous tumor, the complete destruction comprising obtaining a positive slope of the impedance phase diagram (IPS) by analyzing the IPS at end of each time step,
wherein destroying cancer cells of the portion of the cancerous tumor within each time step, plotting the impedance phase diagram at end of each time step, and analyzing the IPS at end of each time step are done in a cycle iteratively after each other.

2. The method of claim 1, wherein analyzing the IPS at end of each time step comprises:
calculating the IPS at end of each time step, the IPS is defined by:
$$IPS = \frac{{Phase}_{2} - {Phase}_{1}}{log ( {Frequency}_{2} ) - log ( {Frequency}_{1} )} , ,$$
wherein Phase₂ is a measured impedance phase value at frequency value of Frequency₂ and Phase₁ is a measured impedance phase value at frequency value of Frequency₁;
comparing the calculated IPS with zero; and
detecting the complete destruction of the portion of the cancerous tumor responsive to the calculated IPS being a positive IPS.

3. The system of claim 2, wherein stopping destroying of the cancer cells comprises ceasing applying the DC voltage between the two electrodes if a value of the calculated IPS is more than zero.

4. The method of claim 1, wherein measuring the set of electrical impedance phase values from the portion of the cancerous tumor at end of each time step comprises:
connecting the two electrodes of the electrical probe to the impedance analyzer device;
applying an AC voltage in a sweeping range of frequencies between 1 Hz and 1 MHz to the two electrodes;
measuring the set of electrical impedance phase values respective to the swept range of frequencies; and
plotting the measured set of electrical impedance phase values versus the swept range of frequencies.

5. The method of claim 1, wherein electrolyzing peripheral medium surrounding the cancer cells of the portion of the cancerous tumor comprises:
connecting the two electrodes of the electrical probe to the DC voltage generator; and
applying a DC voltage with a magnitude between 0.5 V and 10 V between the two electrodes during each time step utilizing the DC voltage generator.

6. The method of claim 5, wherein destroying cancer cells of the portion of the cancerous tumor within each time step further comprises vacuum suction of dead cells, comprising:
connecting a vacuum pump to a proximal end of the electrical probe; and
driving out the dead cells from the portion of the cancerous tumor by applying a vacuum pressure more than 20 KPa to the proximal end of the electrical probe.

7. The method of claim 1, wherein the set of pre-determined time steps comprises at least one of a set of equal time steps, a set of unequal time steps, and combinations thereof.

8. The method of claim 1, wherein each time step of the set of pre-determined time steps comprising a time period in a range between 15 seconds and 3 minutes.

9. The method of claim 1, wherein putting the two electrodes of the electrical probe in contact with the portion of the cancerous tumor comprises:
inserting a distal end portion of a first electrode of the electrical probe into the portion of the cancerous tumor, the first electrode comprising a first electrically conductive needle comprising a hollow needle; and
pushing a distal end portion of a second electrode of the electrical probe through the first needle electrode into the portion of the cancerous tumor, the second electrode comprising a second electrically conductive needle with a nanoporous surface placed inside the first electrode.

10. The method of claim 9, wherein putting the two electrodes of the electrical probe in contact with the portion of the cancerous tumor further comprises identifying a location of the portion of the cancerous tumor by at least one of monitoring inserting the distal end portion of the first electrode of the electrical probe into the portion of the cancerous tumor with guide of sonography imaging, comparing an electrical impedance value of a location of the inserted distal end portion of the first electrode with a reference impedance value, and combinations thereof.

11. The method of claim 10, wherein identifying the location of the portion of the cancerous tumor by comparing the electrical impedance value of the portion of location of the inserted distal end portion of the first electrode with the reference impedance value comprises:
connecting the two electrodes of the electrical probe to the impedance analyzer device;
applying an AC voltage in a sweeping range of frequencies between 1 Hz and 1 MHz to the two electrodes;
measuring an electrical impedance value of the location of the inserted distal end portion of the first electrode at a reference frequency value; and
identifying location of the inserted distal end portion of the first electrode being the location of the portion of the cancerous tumor responsive to the measured electrical impedance value being less than the reference impedance value.

12. The method of claim 11, wherein:
the reference frequency value comprises a frequency value of 1 kHz; and
the reference impedance value comprises an impedance value of 1500Ω.

13. The method of claim 9, further comprising preparing the electrical probe, comprising:
generating a nanoporous surface on an outer surface of the second electrically conductive needle;
coating a second electrically insulating layer on the second electrically conductive needle except a distal end portion of the second electrically conductive needle;
placing the second electrically conductive needle inside the first electrically conductive needle;
covering an outer surface of the first electrically conductive needle with a first electrically insulating layer except a distal end portion of the first electrically conductive needle; and
attaching two electrical connectors to the first electrically conductive needle and the second electrically conductive needle, comprising:
attaching a first electrical connector onto a surface of the first electrically conductive needle adjacent to a proximal end of the first electrically conductive needle; and
attaching a second electrical connector onto a proximal end of the second electrically conductive needle.

14. The method of claim 13, wherein generating the nanoporous surface on the outer surface of the second electrically conductive needle comprises forming a plurality of nanopores, each of the nanopores with a diameter of less than 300 nm.

15. The method of claim 13, wherein generating the nanoporous surface on the outer surface of the second electrically conductive needle comprises wet etching of the outer surface of the second electrically conductive needle, comprising:
placing the second electrically conductive needle in a mixture of HCl and HNO₃ with a weight ratio of (8:1) of (HCl:HNO₃);
heating the mixture of HCl and HNO₃ containing the second electrically conductive needle to a temperature of 60° C.;
maintaining the mixture of HCl and HNO₃ containing the second electrically conductive needle at 60° C. for 180 seconds;
removing the second electrically conductive needle from the mixture of HCl and HNO₃;
washing the second electrically conductive needle with deionized water; and
drying the second electrically conductive needle with compressed air.

16. The method of claim 13, wherein generating the nanoporous surface on the outer surface of the second electrically conductive needle comprises dry etching of the outer surface of the second electrically conductive needle, comprising
placing the second electrically conductive needle in a Reactive Ion Etching (RIE) system; and
RIE processing of the second electrically conductive needle with a processing mixture comprising SF₆ with a flow rate of 150 sccm and O₂ with a flow rate of 150 sccm for 20 minutes at a radiofrequency (RF) power of 250 W and a processing pressure of 20 mTorr.
